(19) United States
(12) Patent Application Publication
(10) Pub. No.: US 2024/0335288 A1
(43) Pub. Date: Oct. 10, 2024

(54) LEAFLET AUGMENTOR

(71) Applicant: Edwards Lifesciences Innovation (Israel) Ltd., Caesarea (IL)

(72) Inventors: Valentina Ruban, Holon (IL); Carmel Peleg, Pardes Hana-Karkur (IL); Yaron Herman, Givat Ada (IL); Anna Khodos, Harish (IL)

(21) Appl. No.: 18/747,863

(22) Filed: Jun. 19, 2024

Related U.S. Application Data
(63) Continuation of application No. PCT/IB2022/062188, filed on Dec. 14, 2022.
(60) Provisional application No. 63/292,353, filed on Dec. 21, 2021.

(51) Int. Cl. A61F 2/24

(52) U.S. Cl. CPC A61F 2/2463; A61F 2/2457; A61F 2220/0008; A61F 2220/0075; A61F 2220/0091; A61F 2250/0012

(57) ABSTRACT

An augmenter (30) for a first leaflet of a heart, the first leaflet having a lip and a root, the valve having a second leaflet. The augmenter comprises a first panel (62), a second panel (64), and an anchor (38) coupled to the first panel. In some applications, the second panel is articulatably coupled to the first panel by an articulatable coupling (66). The augmenter is expandable by increasing an angular disposition between the first panel and the second panel, thereby increasing a dimension of an opening (40) between the panels opposite the articulatable coupling. The augmenter is collapsible by reducing the angular disposition. The anchor is configured to anchor the first panel to the first leaflet such that the articulatable coupling is disposed closer, than the lip and the opening, to the root. Other applications are also described.

### CROSS-REFERENCES TO RELATED APPLICATIONS

This application is a continuation of International Patent Application No. PCT/IB2022/062188, filed Dec. 14, 2022, which claims the benefit of U.S. Patent Application No. 63/292,353, filed Dec. 21, 2021, the entire disclosures all of which are incorporated by reference for all purposes.

### BACKGROUND

Cardiac valves are subject to damage from a variety of causes. Damaged valves may result in regurgitation of blood from the ventricle to the atrium during ventricular systole, eventually causing heart failure. Devices designed to prevent backflow through the damaged valve could be helpful.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

Exemplary applications described herewithin are directed towards systems, apparatuses, devices, and methods to reduce regurgitation through a heart valve by improving coaptation between leaflets of the valve, e.g., by diminishing leaflet flail, prolapse, or abnormal leaflet motion. For example, leaflet augmenters or augmentors are described. The terms augmenter and augmentor are interchangeable as used herein and mean the same thing.

For some applications, a leaflet augmenter or augmentor comprises two articulatably-coupled panels. The first panel is anchorable to a first valve leaflet by anchors, e.g., tissue-piercing elements, T-bars, threads, sutures, pins, clips, or locking elements. The second panel is expandable or collapsible via the articulatable coupling, such that the second panel moves away from or toward the first panel during phases of the cardiac contraction cycle. That is, during ventricular diastole, the second panel moves toward the first panel in response to blood flow through the valve, whereas during ventricular systole, the second panel moves away from the first panel due to increased intraventricular pressure. As the valve closes, the second panel coapts with a second valve leaflet that, prior to implantation of the augmenter, did not coapt properly with the first leaflet.

For some applications, a flexible connector is disposed between the first panel and the second panel, in a manner that limits expansion of the augmenter by limiting the angular extent of an opening between the first panel and the second panel. For some applications, the flexible connector can connect lips of the first and second panels. For some applications, the flexible connector can connect lateral edges of the first and second panels.

The connector can comprise at least one wire, string, or thread. For some applications, the connector comprises a flexible third panel. The third panel can comprise a permeable net, e.g., an open-meshed fabric, a web of interwoven threads, or other substantially open structure. For some applications, the third panel has an accordion fold.

For some applications in which the connector comprises a third panel, the augmenter further comprises a flexible fourth panel, e.g., with each of the third and fourth panels connecting respective lateral edges of the first and second panels. Such an arrangement can stabilize the augmenter and limit the extent of opening between the first panel and the second panel.

The connector can include a cell-growth-inhibiting substance. The connector can include a cell-growth-promoting substance.

The panels can have a substantially rectangular shape or a substantially elliptical shape or can have a different suitable shape.

The panels can comprise a flexible fabric. For some applications, the fabric can have tissue growth-promoting or tissue growth-inhibiting properties. For some such applications, one side of each panel can comprise fabric having different properties than the fabric on the opposite side of the panel, and/or the fabric of one panel can have different properties than the fabric of the other panel.

For some applications, the augmenter comprises a frame, which can be elastic. For applications in which the panels comprise a fabric, the frame can support the fabric. Each panel can comprise a two-ply fabric compartment into which the frame is inserted. The elastic frame can be pretensioned within the fabric frame.

For some applications in which the augmenter comprises a frame, in addition to or alternatively to the augmenter comprising a flexible connector that limits expansion of the augmenter, the frame can itself limit expansion of the augmenter. The articulable coupling can also be part of a frame or frames disposed circumferentially within one or both panels, as described herein below.

The augmenter can be biased toward the first panel and the second panel having a predetermined angular disposition therebetween. For some applications, the coupling between the first and second panels can contribute to limiting the expansion of the augmenter. The coupling can be used to bias the augmenter toward the two panels being in either a collapsed state or an expanded state. For some applications, the stiffness of the articulatable coupling, e.g., a hinge, is biased to respond to blood flow through the valve such the pressure of blood passing from an atrium to a ventricle of a heart during diastole tends to collapse the second panel against the first panel.

For some applications, the augmenter can be biased toward an expanded or collapsed state by means other than the articulatable coupling itself. For example, elasticity and/or shape memory of the frame can provide such biasing.

For some applications, a discrete spring (e.g., a torsion spring, a leaf spring, a cantilever spring, or an arc spring) can provide such biasing—e.g., can provide the augmenter with a clothespin-like characteristic (e.g., biased either open or closed).

For some applications, a leaflet augmenter comprises three panels disposed around a central axis and diverging from each other. For some applications, the panels can be arranged at equal angles, i.e., 120 degrees between each pair. For some applications, the panels can be arranged such that a first and a second panel are disposed at less than 120 degrees from each other, and the third panel at more than 120 degrees from each of the other panels. A first of the three panels is anchorable via an anchor to a first valve leaflet. The augmenter can be configured such that, during systole, one or both of the second and third panels coapts with a second valve leaflet that, prior to implantation of the augmenter, did not coapt properly with the first leaflet.

In accordance with some applications, a system and/or an apparatus, including an augmenter for a first leaflet of a valve of a heart, the first leaflet having a lip and a root, the valve also having a second leaflet. For some applications, the augmenter includes a first panel and a second panel that is articulatably coupled to the first panel by an articulatable coupling.

For some applications, the second panel can be articulatably coupled to the first panel, such that the augmenter is expandable by increasing an angular disposition between the first panel and the second panel, thereby increasing a dimension of an opening defined between the first panel and the second panel opposite the articulatable coupling. For some applications, the augmenter is collapsible by reducing the angular disposition.

For some applications, the augmenter can further include an anchor. For some applications, the anchor is coupled or couplable to the first panel. For some applications, the anchor is configured to anchor the first panel to the first leaflet such that the first panel is disposed against the first leaflet, and the articulatable coupling is disposed closer than the lip, and closer than the opening, to the root.

For some applications, the anchor is configured to anchor the first panel to the first leaflet such that the augmenter expands during ventricular systole and collapses during ventricular diastole.

For some applications, the anchor is configured to anchor the first panel to the first leaflet such that the opening faces the ventricle.

For some applications, the system/apparatus further includes a flexible connector connected to the first panel and to the second panel in a manner that limits expansion of the augmenter by limiting the angular disposition between the first panel and the second panel.

For some applications, the anchor is coupled to the first panel closer to the articulatable coupling than to a lip of the first panel.

For some applications, each of the first panel and the second panel is substantially circular or elliptical in shape.

For some applications, each of the first panel and the second panel is substantially rectangular in shape.

For some applications, the first panel is sufficiently flexible to conform to a shape of the first leaflet.

For some applications, the first panel is dimensioned such that the anchoring, by the anchor, of the first panel to the first leaflet is such that the first leaflet overhangs beyond the lip of the first leaflet.

For some applications, the second panel is dimensioned to overhang beyond an edge of the first panel.

For some applications, the second panel is articulatably coupled to the first panel along an entire length of an edge of each panel.

For some applications, the second panel is articulatably coupled to the first panel along a partial length of an edge of each panel.

For some applications, the articulatable coupling includes stitches.

For some applications, the articulatable coupling includes a hinge.

For some applications, the articulatable coupling includes a flexure.

For some applications, the anchor includes a clip.

For some applications, the anchor includes a tissue-piercing element.

For some applications, the augmenter is configured such that anchoring of the first panel to the first leaflet by the anchor increases an efficiency of the valve in inhibiting regurgitation of blood through the valve during ventricular systole.

For some applications, the first panel is dimensioned such that, upon anchoring of the first panel to the first leaflet by the anchor, the first panel overhangs beyond the lip of the first leaflet.

For some applications, the second panel is dimensioned to overhang beyond an edge of the first panel.

For some applications, the first leaflet is a flailing leaflet, and the augmenter is configured to be anchored to the flailing first leaflet such that the second panel coapts with the second leaflet, thereby inhibiting flailing of the first leaflet.

For some applications, the second leaflet is a flailing leaflet, and the augmenter is configured to be anchored to the first leaflet such that the second panel coapts with the flailing second leaflet, thereby inhibiting flailing of the second leaflet.

For some applications, the augmenter is configured such that, while the first panel is anchored to the first leaflet by the anchor, expansion of the augmenter reduces regurgitation of blood through the valve.

For some applications, the augmenter includes a polymer that is shaped to define the first panel and the second panel.

For some applications, the augmenter includes a silicone that is shaped to define the first panel and the second panel.

For some applications, the system/apparatus further includes a tether coupled to and extending from the first panel and configured to be attached to ventricular tissue while the anchor anchors the first panel to the first leaflet.

For some applications, the anchor is a leaflet anchor, and the system/apparatus further includes a ventricular anchor, coupled to the tether, and configured to anchor the tether to the ventricular tissue while the leaflet anchor anchors the first panel to the first leaflet.

For some applications, the ventricular tissue is tissue of a wall of the ventricle.

For some applications, the ventricular tissue is tissue of a papillary muscle.

For some applications, the ventricular anchor is coupled to the tether in a manner that facilitates adjustment of tension on the tether.

For some applications, the system/apparatus further includes a winch coupled to the ventricular anchor, the anchor being coupled to the tether via the winch such that the tension on the tether is adjustable by actuation of the winch.

For some applications, at least the first panel includes a cell-growth-inducing material.

For some applications, the second panel includes a cell-growth-inducing material.

For some applications, the second panel is formed from a cell growth-inhibiting material.

For some applications, each of the first panel and the second panel include a flexible fabric.

For some applications, the augmenter further includes at least one frame.

For some applications, the at least one frame serves to reinforce the fabric.

For some applications, the fabric of each of the first panel and the second panel is disposed over the at least one frame such that the frame supports the fabric.

For some applications, the frame is shaped to define a hoop.

For some applications, the frame has an H shape, such that the crossbar of the H traverses a face of the panel.

For some applications, the at least one frame is positioned along at least one edge of each panel.

For some applications, the at least one frame includes a first frame and a second frame, the first frame being articulatably coupled to the second frame.

For some applications, the at least one frame includes a single integrated frame, having an articulation site at which the second panel is articulatably coupled to the first panel.

For some applications, the integrated frame includes an elastic material that biases the first panel and the second panel toward assuming a predetermined angular disposition therebetween.

For some applications, the integrated frame includes an elastic material that defines a flexure connection between the first panel and the second panel.

For some applications, the at least one frame is stitched to the fabric.

For some applications, for each of the first panel and the second panel, the flexible fabric defines a first fabric layer disposed over a first side of the at least one frame. For some applications, the panel further includes a second fabric layer disposed over a second side of the at least one frame, such that the frame is sandwiched between the first fabric layer and the second fabric layer.

For some applications, the frame and the fabric are dimensioned with respect to each other such that the frame and the fabric are pretensioned.

For some applications, when the fabric is disposed over the frame, the frame provides a specific form and shape to the panel.

For some applications, the augmenter is biased toward the first panel and the second panel having a predetermined angular disposition therebetween.

For some applications, the augmenter is biased to collapse by reducing the angular disposition between the second panel and the first panel.

For some applications, the augmenter is biased to expand by increasing the angular disposition between the second panel and the first panel.

For some applications, the first panel has a lip opposite the articulatable coupling, and a lateral edge extending between the articulatable coupling and the lip of the first panel. For some applications, the second panel has a lip opposite the articulatable coupling, and a lateral edge extending between the articulatable coupling and the lip of the second panel. For some applications, the opening is defined by the lip of the first panel and the lip of the second panel.

For some applications, the system/apparatus further includes a flexible connector connected to the first panel and to the second panel in a manner that limits expansion of the augmenter by limiting the angular disposition between the first panel and the second panel.

For some applications, the flexible connector is located opposite the articulatable coupling, extending between the first panel and the second panel.

For some applications, the flexible connector connects the lip of the first panel and the lip of the second panel.

For some applications, the flexible connector connects a substantially central part of the lip of the first panel to a substantially central part of the lip of the second panel.

For some applications, the flexible connector is a first flexible connector, and the augmenter further includes a second flexible connector. For some applications, the first flexible connector connects a first lateral part of the lip of the first panel to a first lateral part of the lip of the second panel.

For some applications, the second flexible connector connects a second lateral part of the lip of the first panel, substantially opposite the first lateral part of the lip of the first panel, to a second lateral part of the lip of the second panel, substantially opposite the first lateral part of the lip of the second panel.

For some applications, the flexible connector connects (i) a site on the first panel partway between the articulatable coupling and the lip of the first panel, and (ii) a site on the second panel partway between the articulatable coupling and the lip of the second panel.

For some applications, the flexible connector connects the lateral edge of the first panel to the lateral edge of the second panel.

For some applications, the flexible connector includes a flexible third panel extending between the first panel and the second panel.

For some applications, the flexible connector includes a cell-growth-inhibiting substance.

For some applications, the flexible connector includes a cell-growth-promoting substance.

For some applications, the flexible third panel connects the lip of the first panel to the lip of the second panel.

For some applications, the third panel includes a fabric.

For some applications, the third panel includes a net.

For some applications, the anchor is configured to anchor the first panel to the first leaflet such that the third panel coapts with the second leaflet during systole.

For some applications, the anchor is configured to anchor the first panel to the first leaflet such that the third panel faces the ventricle during systole.

For some applications, the lateral edge of the first panel is a first lateral edge of the first panel. For some applications, the lateral edge of the second panel is a first lateral edge of the second panel, substantially aligned with the first lateral edge of the first panel. For some applications, the first panel has a second lateral edge, substantially opposite the first lateral edge of the first panel, and extending between the articulatable coupling and the lip of the first panel. For some applications, the second panel has a second lateral edge substantially opposite the first lateral edge of the second panel, extending between the articulatable coupling and the lip of the second panel, and substantially aligned with the second lateral edge of the first panel. For some applications, the third panel extends between the first lateral edge of the first panel and the first lateral edge of the second panel.

For some applications, the augmenter further includes a flexible fourth panel, extending between the second lateral edge of the first panel and the second lateral edge of the second panel.

For some applications, the third panel and the fourth panel include folds configured to collapse inward when the opening collapses.

For some applications, the flexible connector includes at least one connector element selected from the group consisting of: a wire, and a thread.

For some applications, the flexible connector includes exactly one connector element selected from the group.

For some applications, the anchor is coupled to the first panel closer to the articulatable coupling than to the lip of the first panel.

For some applications, the anchor includes a toggle anchor.

For some applications, the toggle anchor has a blunt end and a sharp end and is configured to be advanced through the first leaflet sharp-end-first.

For some applications, the anchor is coupled to the first panel by a flexible thread.

For some applications, the anchor is a first anchor, and the augmenter further includes a second anchor that is coupled to the first panel, and that is configured to anchor the first panel to the first leaflet.

For some applications, the first anchor includes a first toggle anchor, and the second anchor includes a second toggle anchor.

For some applications, the augmenter is configured such that, while (i) the first panel is anchored to the first leaflet, and (ii) the articulatable coupling is disposed closer to the root, than the lip and the opening, blood flow in the heart transitions the augmenter toward its expanded configuration during ventricular systole, and toward its collapsed configuration during ventricular diastole.

For some applications, the system/apparatus further includes a flexible connector connected to the first panel and to the second panel in a manner that limits expansion of the augmenter during ventricular systole.

In accordance with some applications, a system and/or an apparatus, including an augmenter for a first leaflet of a valve of a heart, the first leaflet having a lip and a root, the valve having a second leaflet. The system/apparatus includes a first panel, a second panel, a third panel, and/or an anchor.

For some applications, each of the panels has a respective outer edge. For some applications, each of the panels extends inwardly from the outer edge, such that the first, second, and third panels converge at an axis of the augmenter. For some applications, each of the panels defines a panel plane that is substantially parallel with the axis. For some applications, the anchor is configured to anchor the first panel to the first leaflet. For some applications, the augmenter is sterile.

For some applications, the augmenter includes a polymer that is shaped to define the first panel, the second panel, and the third panel.

For some applications, the augmenter includes a silicone that is shaped to define the first panel, the second panel, and the third panel.

For some applications, the anchor is configured to attach the first panel to the first leaflet such that the second panel extends away from the first panel toward the second leaflet.

For some applications, the anchor is configured to anchor the first panel to the first leaflet such that the third panel extends away from the first panel toward the ventricle.

For some applications, the anchor is coupled to the first panel closer to the edge of the first panel than to the axis of the augmenter.

For some applications, each of the first panel, the second panel, and the third panel is substantially circular or elliptical in shape.

For some applications, each of the first panel, the second panel, and the third panel is substantially rectangular in shape.

For some applications, the first panel is sufficiently flexible to conform to a shape of the first leaflet.

For some applications, the first panel is dimensioned such that the anchoring, by the anchor, of the first panel to the first leaflet is such that the third panel overhangs beyond the lip of the first leaflet.

For some applications, the second panel and the third panel collectively define a face, and the anchor is configured to anchor the first panel to the first leaflet such that: (i) the first panel is disposed against the first leaflet, and (ii) the face is presented as a coaptation surface to the second leaflet.

For some applications, the anchor is configured to anchor the first panel to the first leaflet such that the third panel is disposed in the ventricle.

For some applications, the anchor is configured to anchor the first panel to the first leaflet such that, during ventricular systole, the second panel extends away from the first panel and across an upstream surface of the second leaflet.

For some applications, the anchor is configured to anchor the first panel to the first leaflet such that, during ventricular systole, the second panel is disposed against an upstream surface of the second leaflet.

For some applications, the anchor is configured to anchor the first panel to the first leaflet such that, during ventricular systole, a lip of the second leaflet contacts the third panel.

For some applications, the system/apparatus further includes a tether coupled to and extending from the first panel and configured to tether the first panel to ventricular tissue of the heart.

For some applications, the anchor is a leaflet anchor, and the system/apparatus further includes a ventricular anchor, coupled to the tether, and configured to anchor the tether to the ventricular tissue while the leaflet anchor anchors the first panel to the first leaflet.

For some applications, the ventricular tissue is tissue of a wall of the ventricle.

For some applications, the ventricular tissue is tissue of a papillary muscle.

For some applications, the ventricular anchor is coupled to the tether in a manner that facilitates adjustment of tension on the tether.

For some applications, the system/apparatus further includes a winch coupled to the ventricular anchor, the anchor being coupled to the tether via the winch such that the tension on the tether is adjustable by actuation of the winch.

For some applications, at least the first panel includes a cell-growth-inducing material.

For some applications, the second panel includes a cell-growth-inducing material.

For some applications, the third panel includes a cell growth-inducing material.

For some applications, the second panel includes a cell-growth-inhibiting material.

For some applications, the third panel includes a cell growth-inhibiting material.

For some applications, each of the first panel, the second panel, and the third panel includes a flexible fabric.

For some applications, the augmenter further includes a frame.

For some applications, the frame serves to reinforce the fabric.

For some applications, the fabric of each of the first panel, the second panel, and the third panel is disposed over the frame such that the frame supports the fabric.

For some applications, for each of the first, second, and third panels, the frame extends along the outer edge the panel, substantially parallel with the axis.

For some applications, the frame includes a single integrated frame defining an outline of the first panel, the second panel, and the third panel.

For some applications, the integrated frame includes an elastic material that biases the first panel, the second panel, and the third panel toward assuming a predetermined angular disposition with respect to each other.

For some applications, the integrated frame includes an elastic material.

For some applications, the frame is stitched to the fabric.

For some applications, for each of the first panel, the second panel, and the third panel: (i) the flexible fabric defines a first fabric layer, disposed over a first side of the frame, and (ii) the panel further includes a second fabric layer, disposed over a second side of the frame, such that the frame is sandwiched between the first fabric layer and the second fabric layer.

For some applications, the frame and the fabric are sized with respect to each other such that the frame and the fabric are pretensioned.

For some applications, when the fabric is disposed over the frame, the frame provides a specific form and shape to the first panel, the second panel, and the third panel.

In accordance with some applications, a method for use with a real or simulated heart valve, the valve having a first leaflet and a second leaflet, the method including transluminally advancing an augmenter to the heart and anchoring the augmenter to the first leaflet. For some applications, the augmenter includes a first panel, a second panel articulatably coupled to the first panel, and an anchor.

For some applications, the method includes anchoring the first panel to the first leaflet using the anchor, such that the first panel of the augmenter is disposed against the first leaflet. For some applications, the method includes anchoring the first panel to the first leaflet using the anchor, such that the second panel, in response to blood flow in the heart, (i) deflects away from the first panel and toward the second leaflet during ventricular systole, and (ii) deflects away from the second leaflet and toward the first panel during ventricular diastole.

For some applications, anchoring the first panel to the first leaflet includes anchoring the first panel to the first leaflet such that the second panel, in response to blood flow in the heart, coapts with the second leaflet during ventricular systole, thereby reducing regurgitation through the valve.

For some applications, deflection of the second panel during ventricular systole leads to the second panel coapting with the second leaflet.

For some applications, the method further includes tethering the first panel to ventricular tissue using a tether connected to the first panel.

For some applications, the system/apparatus further includes adjusting tension on the tether.

For some applications, tethering the first panel to the ventricular tissue includes anchoring the tether to the ventricular tissue using a tissue anchor attached to the tether.

For some applications, the system/apparatus further includes coupling the tissue anchor to the tether via a winch.

For some applications, the system/apparatus further includes adjusting the tension on the tether by actuating the winch.

For some applications, the method further includes attaching a flexible third panel between the first panel and the second panel.

For some applications, deflection of the second panel during ventricular systole is accompanied by the flexible third panel coapting with the second leaflet.

The above method(s) and steps can be performed on a living animal or on a simulation, such as on a cadaver, cadaver heart, simulator (e.g., with the body parts, heart, tissue, etc. being simulated), etc.

In accordance with some applications, a method for use with a real or simulated valve of a heart, the valve having a first leaflet and a second leaflet, and being disposed between an atrium and a ventricle of the heart, the method including transluminally advancing an augmenter to the heart and anchoring the augmenter to the first leaflet. For some applications, the augmenter includes a first panel, a second panel, a third panel, and an anchor.

For some applications, the method includes anchoring the first panel to the first leaflet.

For some applications, each of the panels has a respective outer edge, extends inwardly from the outer edge, such that the first, second, and third panels converge at an axis of the augmenter, and defines a respective panel plane that is substantially parallel with the axis.

For some applications, anchoring the first panel to the first leaflet includes anchoring the first panel to the first leaflet while the second panel and the third panel are disposed within a delivery tool, and the method further includes subsequently deploying the second panel and/or the third panel from the delivery tool.

For some applications, deploying the second panel and the third panel from the delivery tool includes deploying the second panel and the third panel from the delivery tool such that the second panel and the third panel deflect away from each other.

The above method(s) and steps can be performed on a living animal or on a simulation, such as on a cadaver, cadaver heart, simulator (e.g., with the body parts, heart, tissue, etc. being simulated), etc.

In accordance with some applications, a system and/or an apparatus, including a leaflet augmenter for use at a valve of a heart, the valve having at least a first leaflet and a second leaflet. For some applications, the system/apparatus includes a first panel, and a second panel articulatably connected to the first panel, and a tissue-piercing element coupled to the first panel and configured to anchor the first panel to the first leaflet such that, during ventricular systole, closure of the valve is accompanied by articulation of the second panel away from the first panel and toward the second leaflet.

Any of the above systems, devices, apparatuses, etc. in this summary can be sterilized (e.g., with heat, radiation, ethylene oxide, hydrogen peroxide, etc.), and the methods herein can comprise (or in some additional methods consist of) sterilization of one or more of the systems, devices, apparatuses, etc. herein (e.g., with heat, radiation, ethylene oxide, hydrogen peroxide).

The present invention will be more fully understood from the following detailed description of applications thereof, taken together with the drawings, in which:

### BRIEF DESCRIPTION OF THE DRAWINGS

FIGS. 1A-C are schematic illustrations of a native heart valve of a subject;

FIGS. 2A-F, 3A-F, 4A-F, 5A-F, and 6A-C are schematic illustrations of exemplary applications of a leaflet augmenter, in accordance with some applications;

FIGS. 7-8 are schematic illustration of exemplary applications of systems comprising a leaflet augmenter implanted at a native valve of a heart, in accordance with some applications;

FIGS. 9A-B are schematic illustrations showing a leaflet augmenter implanted at a native valve of a heart, in accordance with some applications;

FIGS. 10A-C are schematic illustrations of a leaflet augmenter according to some applications; and

FIG. 11 is a schematic illustration showing a leaflet augmenter implanted at a valve of a heart, in accordance with some applications.

### DETAILED DESCRIPTION

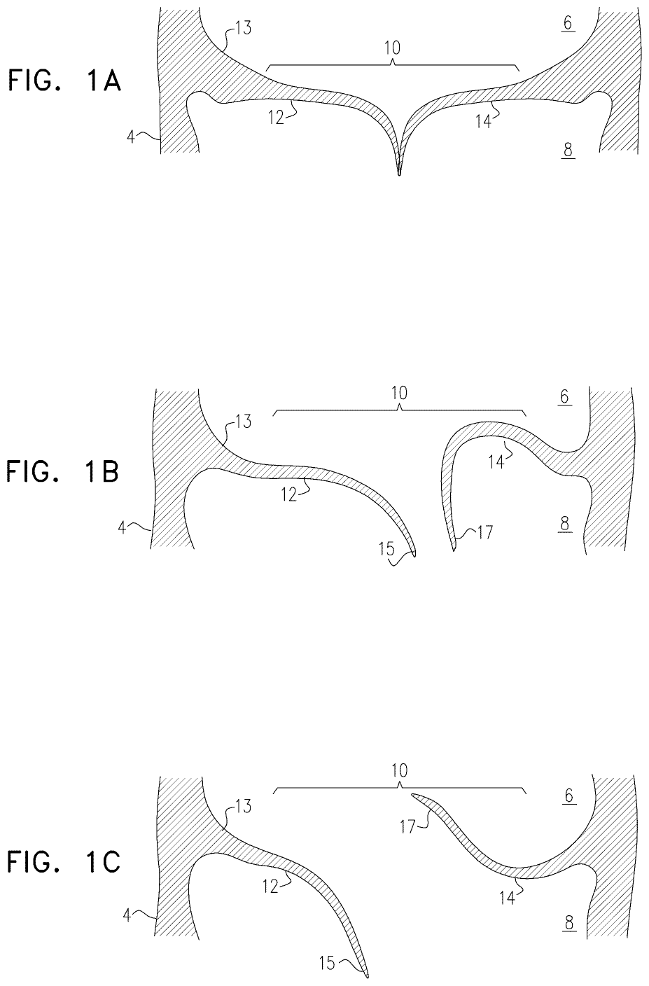

Reference is now made to FIGS. 1A-1C, which are schematic illustrations of part of a native valve 10 of a heart 4 of a subject, in various conditions. FIG. 1A represents a heart having normal and/or healthy anatomy and function. Valve 10 includes a first leaflet 12 and at least a second leaflet 14, each of which has a root 13 at which it is attached, e.g., to an annulus of the valve. For simplicity, only root 13 of first leaflet 12 has been labeled. Valve 10 can be located between an atrium 6 and a ventricle 8 of the heart—i.e., can be an atrioventricular valve such as a mitral valve or a tricuspid valve. In some applications, valve 10 may be a valve between a chamber of a heart and a major blood vessel leading into or out of the chamber. FIG. 1A shows heart 4 in ventricular systole, during which leaflets 12 and 14 coapt, thereby preventing backflow of blood from ventricle 8 into atrium 6.

FIGS. 1B and 1C show heart 4 having a valve 10 in which leaflets 12 and 14 fail to close completely during ventricular systole—i.e., the leaflets fail to completely coapt with each other. The cause of inadequate closure may be valve prolapse or other damage or structural change to the leaflets, roots, chorda tendinae, or annulus of the valve. As a result, regurgitation through valve 10 occurs during ventricular systole. FIG. 1B shows leaflet 14 not reaching leaflet 12, whereas FIG. 1C shows lip 17 of leaflet 14 flailing upward into atrium 6. Failure of leaflets 12 and 14 to coapt renders valve 10 ineffective at preventing regurgitation of blood from ventricle 8 to atrium 6 during ventricular systole.

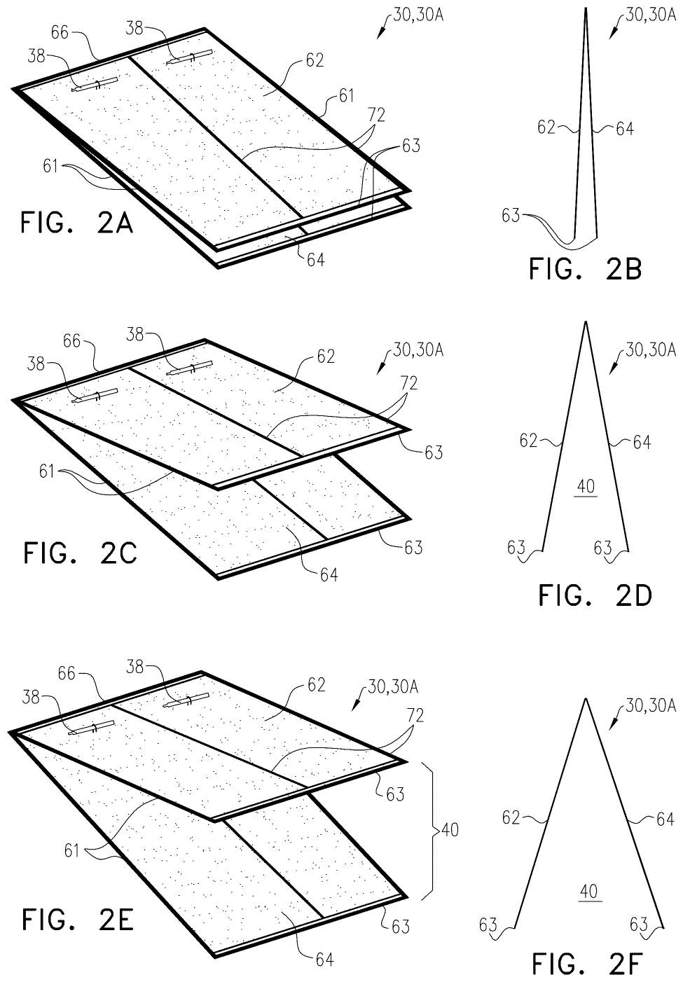

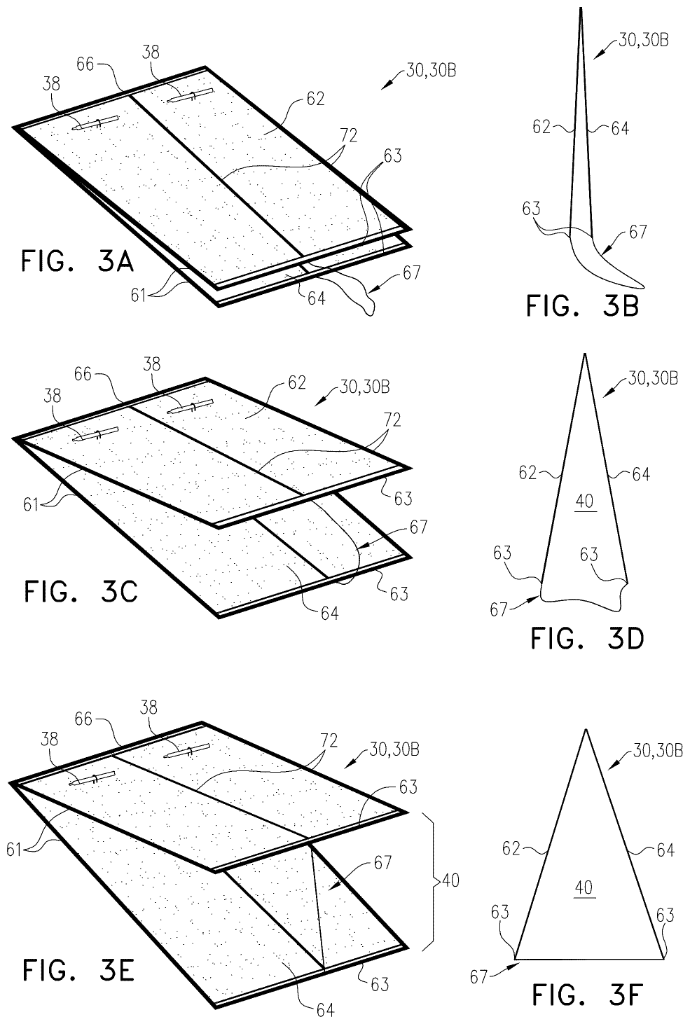

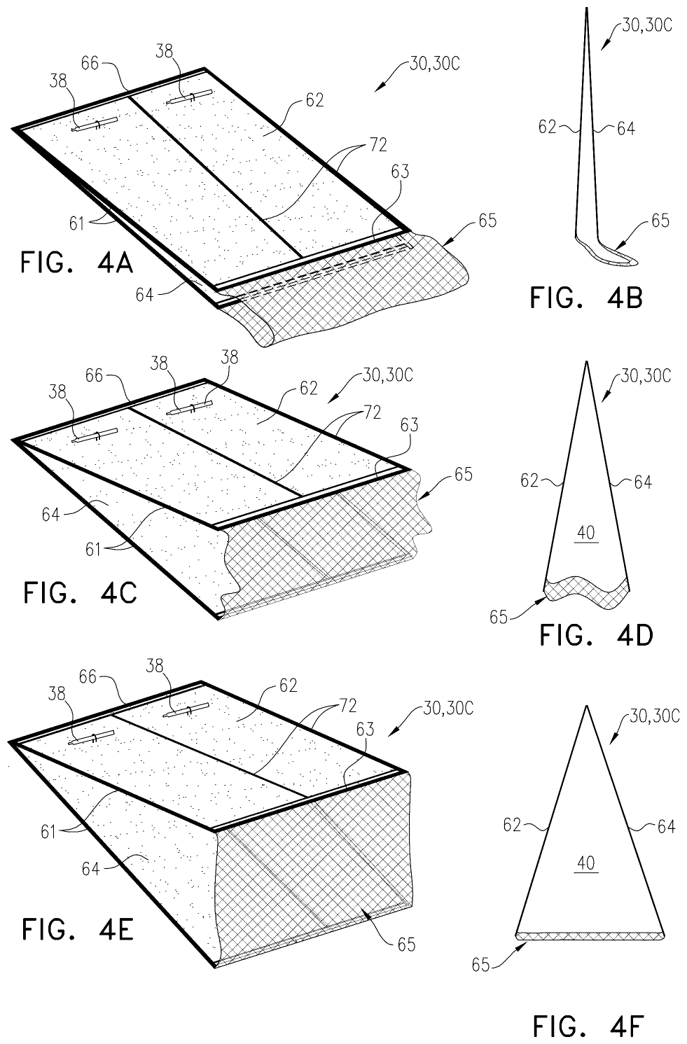

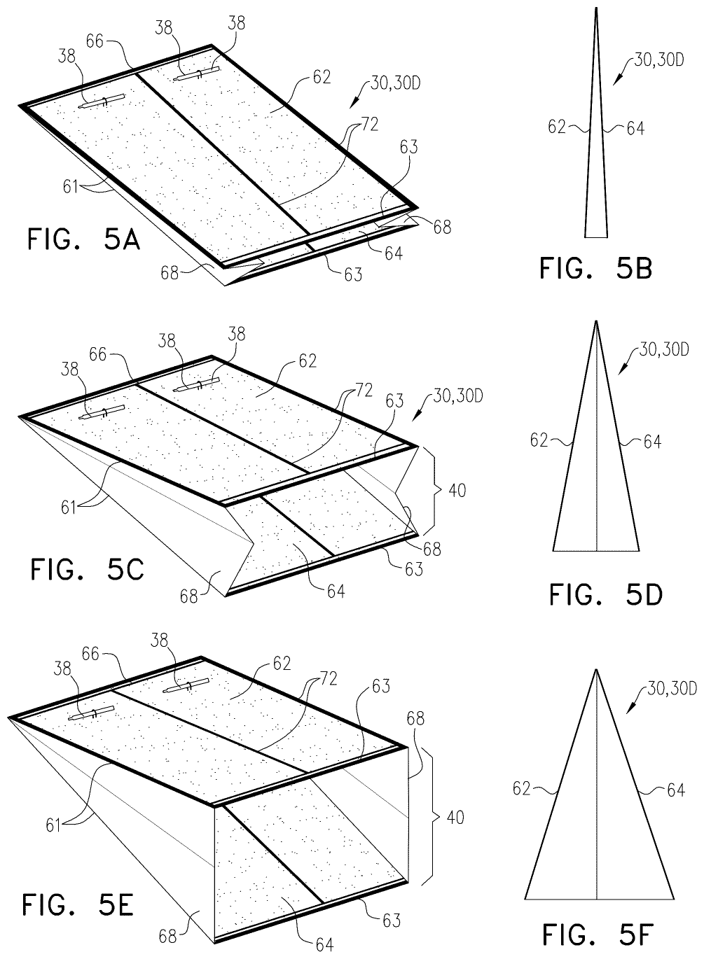

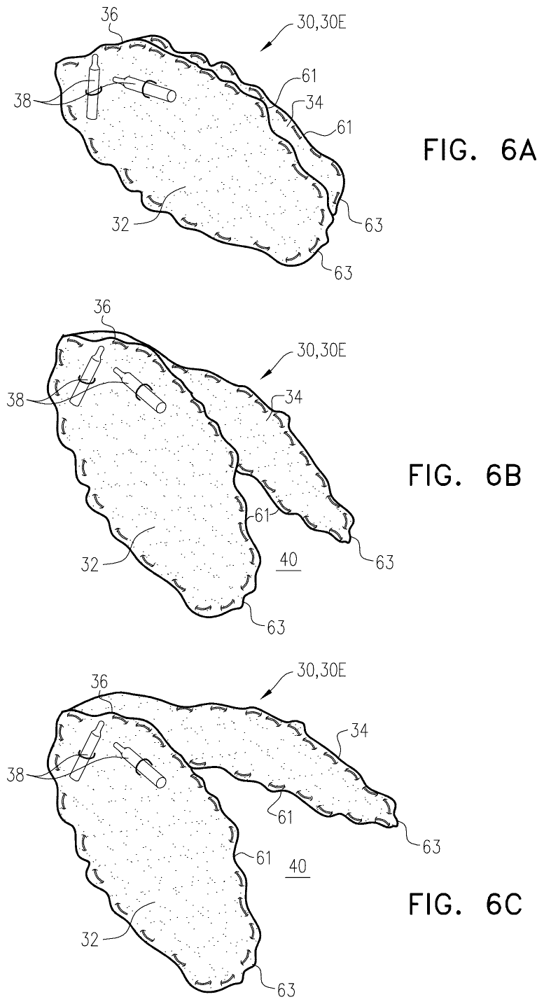

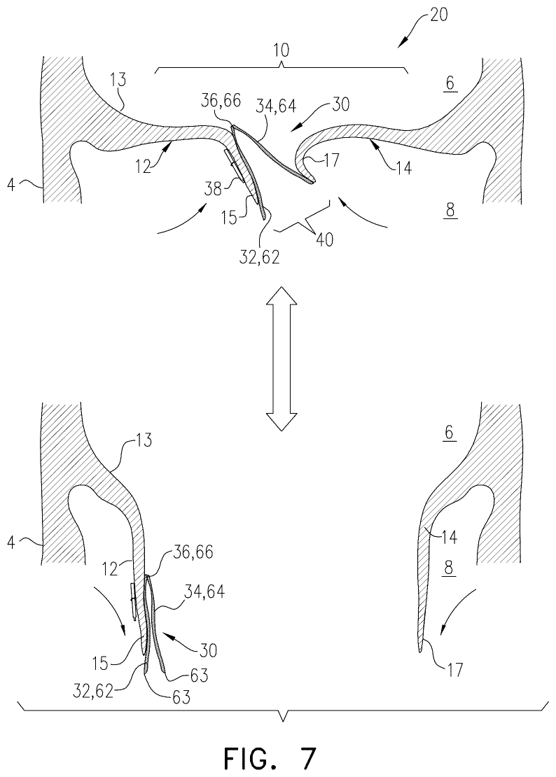

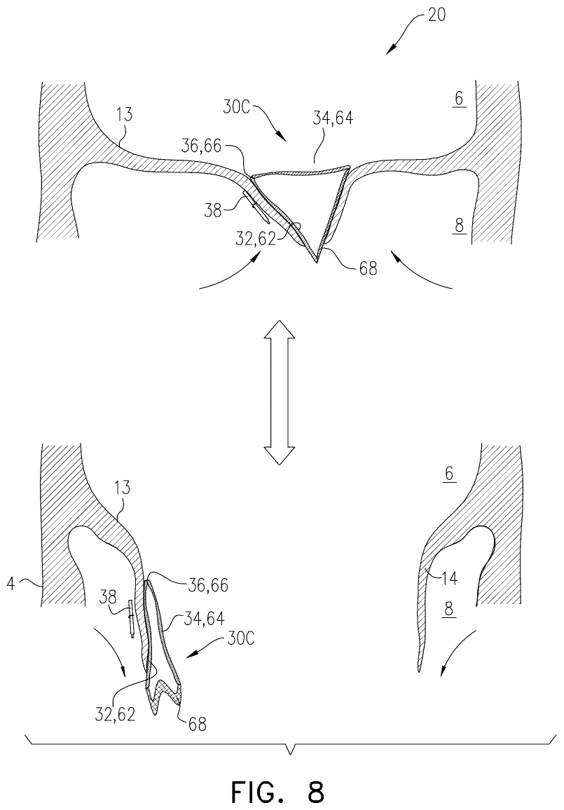

Reference is made to FIGS. 2A-F, 3A-F, 4A-F, 5A-F, and 6A-C, which are schematic illustrations of various variants of a leaflet augmenter or augmentor 30, in accordance with some applications. FIGS. 2A-F show an augmenter 30A, FIGS. 3A-F show an augmenter 30B, FIGS. 4A-F show an augmenter 30C, FIGS. 5A-F show an augmenter 30D, and FIGS. 6A-C show an augmenter 30E, in accordance with some applications. Exemplary variants of leaflet augmenter 30, e.g., as implanted into the heart, are subsequently shown in FIGS. 7, 8, and 9A-B. It is to be understood that the augmenter 30 in FIGS. 7, 8, and/or 9-B can represent any one of the variants (30, 30A, 30B, 30C, 30D, or 30E) shown in FIGS. 2A-6C.

Each of augmenters/augmentors 30A, 30B, 30C, and 30D comprises panels 62 and 64, and augmenter 30E comprises panels 32 and 34. In each variant, panels 62 and 64, or panels 32 and 34, are coupled at an articulatable coupling 66 along one adjacent/parallel edge. In the examples shown, each of panels 62 and 64 has a substantially rectangular shape, and each of panels 32 and 34 has a substantially elliptical shape. In the following descriptions, reference to panels 62 and 64 can be understood to apply also to panels 32 and 34.

Each of panels 62 and 64 can comprise a flexible material. In some applications, each panel can have single layer of the flexible material. In some applications, the panels comprise a double layer of a biocompatible fabric or mesh webbing. The dimensions of panels 62 and 64 can be provided according to the size of the valve and/or of a regurgitating part of the valve that the augmenter 30 is intended to repair.

The articulatable coupling 66 between panels 62 and 64 is such that augmenter 30 is expandable by increasing an angular disposition between the panels, and collapsible by reducing the angular disposition. Expansion of the augmenter increases a dimension of an opening 40 defined between the panels, opposite the articulatable coupling, and collapsing of the augmenter decreases the dimension of the opening.

Each panel (e.g., the flexible material thereof) can be endowed with cell growth-modulating properties. In some applications, the fabric can have tissue growth-promoting or tissue growth-inhibiting properties. For some such applications, one side of each panel can comprise fabric having different properties than the fabric on the opposite side of the panel, and/or the fabric of one panel can have different properties than the fabric of the other panel.

FIGS. 2A-B, 3A-B, 4A-B, 5A-B, and 6A show augmenter 30 (e.g., various examples thereof) in a state in which panels 62 and 64 have a minimal angular separation between them—e.g., a collapsed state. In the collapsed state, lips 63 can be adjacent to each other (and even in contact with each other) and augmenter 30 can appear substantially as a single panel, e.g., panels 62 and 64 can behave substantially as a single panel.

FIGS. 2E-F, 3E-F, 4E-F, 5E-F, and 6C show augmenter 30, e.g., various examples thereof, in a state in which panels 62 and 64 have a maximal angular separation between them—e.g., the largest angular separation allowed by the configuration of the augmenter. Expansion of the augmenter can be limited by articulatable coupling 66 or can be limited by a flexible connector (e.g., as described hereinbelow).

FIGS. 2C-D, 3C-D, 4C-D, 5C-D, and 6B show augmenter 30 (e.g., various examples thereof) in an intermediate state—e.g., via which the augmenter can transition between the collapsed and expanded states.

Coupling 66 between panels 62 and 64 can comprise, for example, a hinge, stitching, a flexure, metal formed in a specific configuration, e.g., nitinol having a specific shape and memory, or other flexible closure, i.e., another means of articulatably coupling the two panels along one edge. The articulatable coupling 66 can connect panels 62 and 64 along the entire length of an edge of each panel. In some cases, the panels are articulatably coupled along a partial length of an edge of each panel.

In some applications, the articulation is biased such that panel 62 and panel 64 have a predetermined angular disposition therebetween, i.e., the coupling can be designed with a stiffness that tends to hold the two panels a predetermined distance apart under conditions of no pressure. The augmenter 30 can be biased to collapse by reducing the angular disposition between the second panel and the first panel. The augmenter 30 can be biased to expand by increasing the angular disposition between the second panel and the first panel. In some cases, the predetermined angular disposition can be greater than 10 degrees and/or less than 120 degrees, e.g., 10-120 degrees (e.g., 20-90 degrees, such as 20-60 degrees) —i.e., such that the augmenter is biased to expand or open.

Bias towards collapse may be desirable in cases in which, e.g., the valve is stenotic, or the ventricle is small e.g., so as to maintain the effective valve orifice as open as possible during diastole (e.g., such that the augmenter does not substantially inhibit blood flow during diastole). Bias toward expansion may be advantageous in cases in which it is particularly challenging to achieve coaptation during diastole, e.g., a large gap exists between the leaflets of the valve, and/or hemodynamics of the heart of the subject are not optimal for blood-flow-induced opening of the augmenter. Thus, the advantage of using an augmenter that is biased toward either collapse or expansion may depend on factors such as the anatomical features and pathology of the valve and heart under treatment.

In some applications, the first and second panels of augmenter 30 can be substantially circular or elliptical in shape, e.g., as shown for panels 32 and 34 of augmenter 30E. In some applications, the panels can be substantially rectangular in shape, e.g., as shown for panels 62 and 64 of augmenters 30A, 30B, 30C, and 30D. It is to be noted that the scope of the present disclosure also includes panels of any other suitable shape.

Panels of any shape can comprise a flexible fabric, or can comprise a plastic or flexible substance, e.g., a silicone or a polymer, that is shaped to define the first panel and the second panel. Panels 62 and 64 can be sufficiently flexible to conform to the shape of the leaflet, i.e., the two panels can be shaped to conform to the outline of a valve leaflet, or they can have a different shape. Panel 62 can be dimensioned to overhang beyond lip 15 of leaflet 12, e.g., as further described in FIGS. 7, 8, and 9A-B hereinbelow.

Panels 62 and 64 can be self-supporting, such that a flexible connector to limit opening 40 is not necessary. Panels 62 and 64 can be reinforced with a frame 72 (or a pair of frames), e.g., with metal inserts, strips of metal such as nitinol, or metal wire. The frame can be woven onto or into the fabric of the panels. In some applications, a stiffening mechanism can be metal threads or fine strips woven into the fabric.

In some applications, and as shown, frame 72 extends along one or more edges and/or lips of the panels. For some such applications, and as shown, frame 72 can span across a central area of each panel, between coupling 66 and lip 63—e.g., such that the frame defines an H shape. Each panel of augmenter 30 can comprise a biocompatible fabric in two layers, between or within which a wire loop is fastened, the wire loop providing flexible stiffness to the panel.

The flexible fabric can define a first fabric layer, disposed over a first side of frame 72. The panel can further comprise a second fabric layer, disposed over a second side of frame 72, such that the frame is sandwiched between the first fabric layer and the second fabric layer. The fabric layers on each side of each panel can comprise the same or different types of fabric. The different types of fabric can differ, e.g., in their tissue growth-promoting properties.

Augmenter 30 can comprise one or more anchors 38, configured to anchor panel 62 (and thereby the augmenter as a whole), to a first leaflet 12 of a valve, as demonstrated in FIGS. 7-9. For example, and as described in more detail hereinbelow, anchors 38 can be configured (e.g., positioned with respect to panel 62) to anchor panel 62 to leaflet 12 such that articulatable coupling 66 is disposed closer to root 13 than is either lip 63 or opening 40.

Anchors 38 can comprise, for example, pins, tissue-piercing elements, T-bars, threads, clips, sutures, or locking elements.

In some applications, anchors 38 can be provided and/or introduced separately, and subsequently attached to panel 62 (e.g., during implantation of augmenter 30). In some applications, augmenter 30 can be provided with anchors 38 pre-attached to panel 62.

The location at which anchor 38 is attached to panel 62 can be near coupling 66—e.g., nearer to the coupling than to lip 63, such as being substantially at coupling 66. Anchors 38 can be flexibly connected to panel 62 such that the anchors are movable and can be directed to penetrate a valve leaflet. In some cases, the anchors are toggle anchors having one end sharper than the other, i.e., a sharp end used to pierce leaflet 12, and a blunt end. The anchors can be attached to the augmenter, i.e., to panel 62, via a flexible thread, string, or wire woven into panel 62.

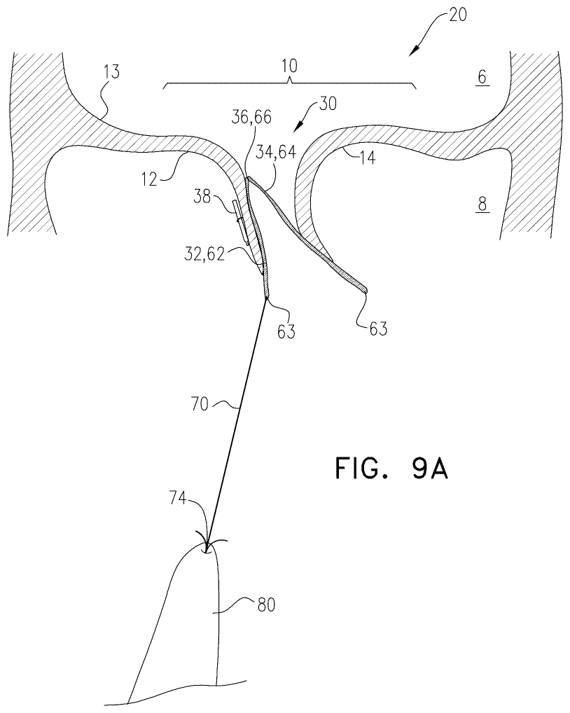

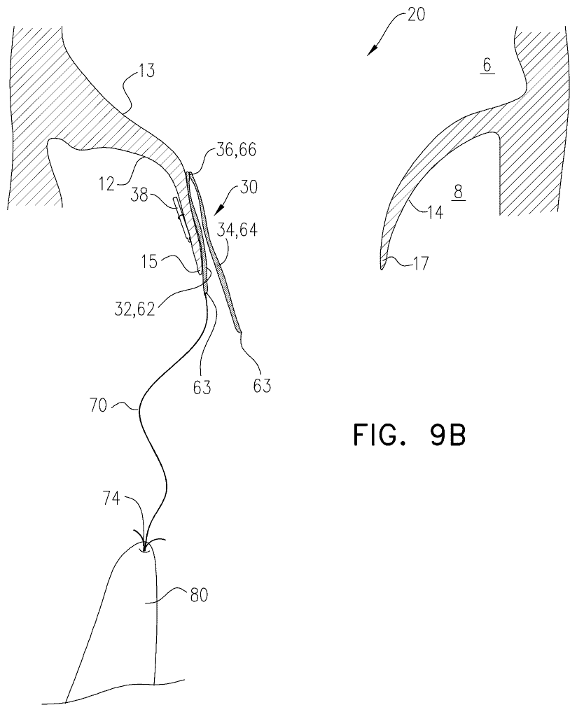

In some applications, panels 62 and 64 are substantially the same size and/or shape. In some applications, panel 64 is a different size (e.g., a different length) than panel 62. For example, panel 64 can be larger (e.g., longer) than panel 64, such that panel 64 extends beyond the lip 63 of panel 62—e.g., as described hereinbelow with reference to FIG. 9A. In some applications, panel 62 can extend beyond lip 63 of panel 64.

FIGS. 2A to 2F show an augmenter 30A, which is a variant of augmenter 30, in accordance with some applications. FIGS. 2A, 2C, and 2E show augmenter 30A in a sequence of conformations from closed (FIG. 2A) to partially open (FIG. 2C) to maximally open (FIG. 2E), as viewed isometrically. FIGS. 2B, 2D, and 2F show the same sequence of conformations from closed to maximally open, as viewed in two dimensions from one side of the augmenter. In general, FIGS. 2A and 2B can be considered to represent the conformation of augmenter 30A during diastole (after the augmenter has been implanted), and FIGS. 2E and 2F can be considered to represent its conformation during systole.

In FIGS. 2A-F, frame 72 serves to support and/or strengthen panels 62 and 64, e.g., providing shape and strength. Frame 72 can be a single integrated frame, defining articulatable coupling 66; in general, frame 72 is referred to herein in the singular. However, in some applications, discrete frames can be provided for each panel of augmenter 30—and these discrete frames can be articulatably coupled to each other at coupling 66. It is to be noted that in applications in which a discrete frame 72 is provided for each panel of augmenter 30A, the frames are coupled only at articulatable coupling 66.

In contrast to applications 30B, 30C, and 30D shown in FIGS. 3A-F to 5A-F, respectively, augmenter 30A lacks a discrete flexible connector between panels 62 and 64 to limit the angular expansion. Rather, frame 72 itself can provide limiting functionality with regard to angular expansion of augmenter 30A. In FIGS. 2A-2F, frames 72 are coupled only at articulatable coupling 66.

FIGS. 3A to 3F show an augmenter 30B, which is a variant of augmenter 30, in accordance with some applications. FIGS. 3A, 3C, and 3E show augmenter 30B in a sequence of conformations from closed (FIG. 3A) to partially open (FIG. 3C) to maximally open (FIG. 3E), as viewed isometrically. FIGS. 3B, 3D, and 3F show the same sequence of conformations from closed to maximally open, as viewed in two dimensions from one side of the augmenter. In general, FIGS. 3A and 3B can be considered to represent the conformation of augmenter 30B during diastole (after the augmenter has been implanted), and FIGS. 3E and 3F can be considered to represent its conformation during systole.

Augmenter 30B further comprises a flexible connector 67 such as a string, thread, or wire. Connector 67 is attached to, and between, lip 63 of panel 62 and lip 63 of panel 64, opposite the articulated coupling 66. Connector 67 constrains the angular disposition between panels 62 and 64 to the extent of opening 40. Connector 67 can be sized as required according to a desired maximal angular disposition between panels 62 and 64, defining the extent of opening 40 when the device is in an open configuration.

During systole, connector 67 prevents panel 64 from flapping wider than the predetermined or desired extent of opening 40. During diastole, when panels 62 and 64 tend to collapse toward each other, connector 67 is not under tension and can hang from the lips of panels 62 and 64. In some applications, more than one connector can be attached between panels 62 and 64, e.g., a connector on each corner framing opening 40, or several connectors along lips 63 of panels 62 and 64. Similarly, in some applications one or more connectors 67 can connect lateral sides 61 of panels 62 and 64. Furthermore, in some applications one or more connectors 67 can connect other regions of the panels—e.g., the middle of each panel (e.g., compared to the position of connector 67 in FIGS. 3A-F, closer to articulatable coupling 66). The number and material composition of the connectors may depend on the desired strength of the connector. It may also depend on the size and shape of the panels, and in some cases, on the type of articulation.

FIGS. 4A to 4F show an augmenter 30C, which is a variant of augmenter 30, in accordance with some applications. As in FIGS. 2 and 3, FIGS. 4A, 4C, and 4E show augmenter 30C in a sequence of conformations from closed (FIGS. 4A, 4B) to partially open (FIGS. 4C, 4D) to maximally open (FIGS. 4E, 4F), as viewed isometrically. FIGS. 4B, 4D, and 4F show the same sequence of conformations from closed to maximally open, as viewed in two dimensions from one side of the augmenter. In general, FIGS. 4A and 4B can be considered to represent the conformation of augmenter 30B during diastole (after the augmenter has been implanted), and FIGS. 4E and 4F can be considered to represent its conformation during systole.

In FIGS. 4A-F is shown that the augmenter can further comprise a third panel 65 of a flexible material. As well as being flexible, panel 65 can be highly permeable—i.e., allows blood to flow therethrough. Third panel 65 can comprise a variety of flexible materials having openings or holes of a predetermined size, e.g., a mesh fabric or net of metal wire or interwoven threads. In some applications, the holes are of such size as to allow free passage of blood therethrough. Panel 65 can comprise a tissue growth-inhibiting substance.

The third panel 65 is disposed along and between lips 63 of panel 62 and panel 64, connecting the edges of panel 62 and panel 64 opposite the articulation, typically spanning opening 40. Panel 65 attaches to lips 63 of panels 62 and 64 opposite the hinge, providing a means of limiting the flap of the augmenter to a predetermined angular opening. Therefore panel 65 can be considered to be a flexible connector, e.g., functioning similarly to flexible connector 67 described hereinabove, mutatis mutandis. Panel 65 can be sized as required according to a desired maximal angular disposition between panels 62 and 64, defining opening 40 when the device is in an open configuration.

Alternatively or additionally, panel 65 can be designed to coapt with the second leaflet 14, providing support for the second leaflet and preventing it from flailing into the atrium during ventricular systole (e.g., as described with reference to FIG. 8).

FIGS. 5A to 5F show an augmenter 30D, which is a variant of augmenter 30, in accordance with some applications. FIGS. 5A, 5C, and 5E show augmenter 30D in a sequence of conformations from closed (FIGS. 5A, 5B) to partially open (FIGS. 5C, 5D) to maximally open (FIGS. 5E, 5F), as viewed isometrically. FIGS. 5B, 5D, and 5F show the same sequence of conformations from closed to maximally open, as viewed in two dimensions from one side of the augmenter. In general, FIGS. 5A and 5B can be considered to represent the conformation of augmenter 30D during diastole (after the augmenter has been implanted), and FIGS. 5E and 5F can be considered to represent its conformation during systole.

Augmenter 30D comprises panels 62 and 64, and articulation 66. In some applications, panels 62 and 64 are supported, or their angular separation is constrained, by one or more third panels 68, e.g., accordion folds, flexible fabric, net, or other flexible sheet, connecting the lateral sides of panels 62 and 64. Panels 68 can be disposed perpendicular to articulation 66. Panels or folds 68 provide stability to the augmenter and frame opening 40 opposite the articulation. When completely closed, as in FIGS. 5A-5B, the accordion folds 68 are fully collapsed or folded (e.g., between panels 62 and 64), such that panels 62 and 64 are nearly adjacent. When fully open, as in FIGS. 5E-5F, the extent of opening 40 can be limited by the size of the folds or additional panels 68 on either side. Therefore panels 68 can be considered to be flexible connectors, e.g., functioning similarly to flexible connector 67 described hereinabove, mutatis mutandis. Panels 68 can be sized as required according to a desired maximal angular disposition between panels 62 and 64, creating opening 40 when the device is in an open configuration. In some applications, the folds comprise flexible fabric that can crumple or balloon outward upon collapse or closure of augmenter 30D.

FIGS. 6A to 6C show an augmenter 30E, which is a variant of augmenter 30, in accordance with some applications. Augmenter 30E is shown in a sequence of conformations from closed (FIG. 6A) to partially open (FIG. 6B) to maximally open (FIG. 6C), as viewed isometrically. In general, FIG. 6A can be considered to represent the conformation of augmenter 30E during diastole (after the augmenter has been implanted), and FIG. 6C can be considered to represent its conformation during systole.

Augmenter 30E has a shape in which panels 32 and 34, corresponding to panels 62 and 64 in applications 30A to 30D, have an elliptical (e.g., circular) outline. The panels are stitched together along one region, creating an articulation 36. As in the applications shown in FIGS. 2A-F to 5A-F, opening 40 can be a free space or opening at the lips 63 of panels 32 and 34. Augmenter 30E can be self-supporting, or can be collapsed or restrained in dimension of angular opening by a string, wire, net, or lateral accordion folds, or any of the other mechanisms for the flexible connector mentioned in the descriptions of FIGS. 2 to 5. That is, descriptions of panels 62 and 64 apply to panels 32 and 34 as well. In applications in which a flexible connector is included, the connector can be sized as required according to a desired maximal angular disposition between panels 32 and 34, creating opening 40 when the device is in an open configuration.

Panels 32 and 34 can comprise a flexible fabric. The fabric can be disposed over and supported by a frame (not shown) comprising metal, plastic, or other material with bendable stiffness, such that the frame supports the fabric. In some applications, each panel can have its own frame, the individual frames articulatably coupled to each other. In some applications, a single integrated frame that extends between both panels and has an articulation site, can be used. Each panel can comprise two layers of the fabric, sandwiching the frame therebetween. In some applications, the frame can be held within the two layers of fabric without being directly stitched to the panel itself (e.g., within each panel, the frame is constrained within a pocket defined by the two layers of fabric). In some applications, the frame can be stitched directly to the fabric.

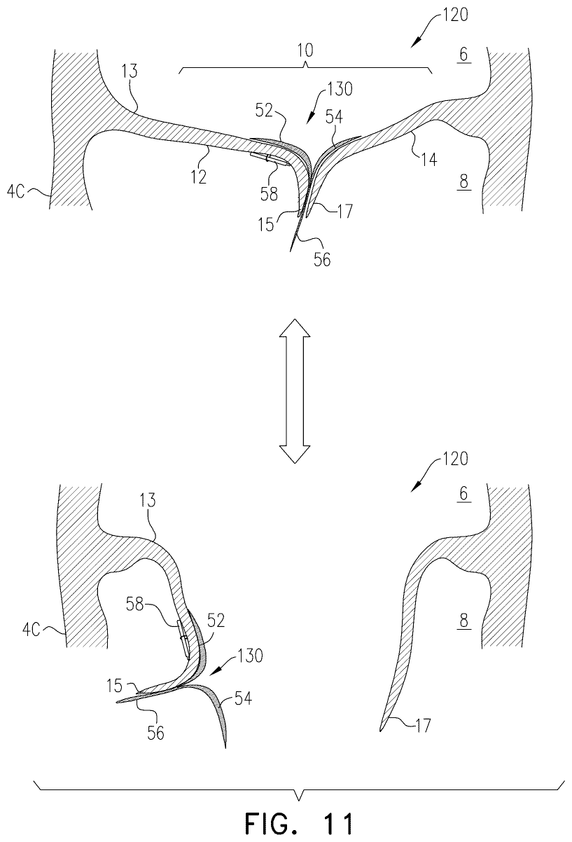

Reference is made to FIGS. 7, 8, and 9A-B, which are schematic illustrations of a system 20 in which augmenter 30 (e.g., a variant thereof) is implanted at valve 10, in accordance with some applications. Applications of the system can be used to treat cases of valve dysfunction, e.g., prolapse, regurgitation, or leaflet flail. Augmenter 30 can be anchored to leaflet 12, or in some applications, to leaflet 14. In some applications, augmenter 30 is anchored to a dysfunctional leaflet. In some applications, augmenter 30 is anchored to a healthy leaflet (e.g., opposite a dysfunctional leaflet). In some applications, a respective augmenter 30 is anchored to each of the leaflets. In each figure, the upper image represents valve 10 of heart 4 during systole, and the lower image represents the valve during diastole. These conventions apply also to FIG. 11, described herewithin below, for augmenter 130.

Although the following description refers to panels 62 and 64, the description is equally applicable to variants of augmenter 30 that comprise panels 32 and 34, mutatis mutandis.

FIG. 7 shows system 20 after augmenter 30 has been transluminally advanced to heart 4, and panel 62 has been anchored to leaflet 12 using anchor 38. Augmenter 30 can be any one of augmenter 30A, 30B, 30C, 30D, or 30E, from FIGS. 2A-F to 6A-C, or a combination of these variants.

Augmenter 30 is configured to be secured to leaflet 12, such as via an anchor 38—e.g., as shown in FIGS. 7, 8, and 9A-B. Augmenter 30 can be secured to either a healthy leaflet (e.g., as shown in FIG. 7), or to a dysfunctional leaflet (e.g., as shown in FIG. 8). Whereas augmenter 30 is shown secured to leaflet 12 in each of FIGS. 7, 8, and 9A-B, different clinical scenarios are illustrated and described in these figures. That is, in some applications, the augmenter can be secured to the more or most dysfunctional leaflet, and in some cases, it can be secured to the leaflet opposite a leaflet having dysfunctional performance. To which leaflet the augmenter is secured depends on the performance of the valve during systole, and which leaflet, or which scallop or segment of that leaflet, can have the greatest need of reinforcement. In some applications, more than one augmenter can be employed to treat a given valve.

Anchor 38 is used to connect panel 62 to leaflet 12. In some applications, and as shown, this anchoring is such that articulatable coupling 66 is disposed between the root 13 and lip 15 of the leaflet—i.e., closer than the lip to the root. In some applications, augmenter 30 can be anchored such that articulatable coupling 66 is disposed at an annulus of valve 10. In some applications, the augmenter 30 can be of a size such that panel 62 covers most of leaflet 12. In some applications, augmenter 30 can cover only a region close to lip 15 of leaflet 12, and/or can cover only one scallop of the leaflet.

In some applications, the first and second panels are the same size and shape (e.g., as shown hereinabove). In some applications, either panel can be larger than the other in at least one dimension, e.g., such that a lip 63 of panel 64 opposite the hinge extends beyond the opposing lip 63 of panel 62, as shown in FIGS. 9A-B.

Panel 62 can be flush with the surface of first leaflet 12, and can comprise a material configured (e.g., sufficiently flexible) to follow the contours of the first leaflet. In some instances, the first panel conforms to the changing form of the first leaflet as it opens and closes during diastole and systole.

In some applications, augmenter 30 is provided with anchor(s) 38 pre-attached to panel 62—e.g., as shown.

In some applications, anchor(s) 38 are provided as discrete elements to be introduced (e.g., coupled to panel 64) during implantation. The anchor is designed to attach or to be coupled to the first panel, generally near articulatable coupling 66, or at least closer to the articulatable coupling than to lip 63.

In FIG. 7, system 20 is shown treating a valve 10 of heart 4 of a subject, the valve having a dysfunction, e.g., such as that shown in FIG. 1B in which leaflet 14 fails to coapt normally with leaflet 12. During systole, augmenter 30 bridges a potential gap between leaflets 12 and 14, such that leaflet 14 contacts panel 64 of augmenter 30. During systole, augmenter 30 is in an open or expanded configuration, such that opening 40 faces ventricle 8, panel 62 is attached to leaflet 12, and panel 64 coapts with leaflet 14. During ventricular diastole, shown in the lower image of FIG. 7, as leaflets 12 and 14 separate (allowing blood into the ventricle 8), panels 62 and 64 of augmenter 30 collapse toward each other, minimizing the opening between them, and thereby reducing obstruction of blood flow through valve 10 (e.g., compared to if the augmenter were to remain in its expanded state).

FIG. 8 shows augmenter 30C having been implanted such that, at least during ventricular systole, third panel 65 becomes disposed against leaflet 14. Opening 40 faces ventricle 8—e.g., both during ventricular systole and during ventricular diastole. During ventricular systole, represented in the upper image of FIG. 8, panel 64 of augmenter 30 coapts with the upstream surface of leaflet 14. Coaptation closes the opening between leaflets 12 and 14, preventing backflow of blood from ventricle 8 into atrium 6. During ventricular diastole, shown in the lower image of FIG. 8, as leaflets 12 and 14 separate (allowing blood into the ventricle 8), panels 62 and 64 of augmenter 30 collapse toward each other, minimizing the opening between them, and thereby reducing obstruction of blood flow through valve 10 (e.g., compared to if the augmenter were to remain in its expanded state).

Reference is now made to FIGS. 9A and 9B, showing additional and separate features present in some applications. Augmenter 30 can further comprise a tether 70 made of a metal wire, suture, fabric thread, or other connecting filament configured to tether panel 62 to ventricular tissue 80. The tether 70 is coupled to and extends from panel 62 and is attached to ventricular tissue 80 via a ventricular anchor 74, while anchor 38 anchors panel 62 to leaflet 12. Anchor 74 can anchor tether 70 to a papillary muscle, ventricular wall, interventricular septum, or other tissue of the ventricle. Tether 70 can be attached to lip 63 of panel 62, or elsewhere on panel 62.

In some applications, the tether is hypothesized to advantageously stabilize the augmenter by regulating the movement of the first panel 32 with respect to the first leaflet 12, providing additional support to the augmenter. The tether can be used to stabilize or further anchor the augmenter in place, e.g., in the case of a torn chordae tendinae. Tether 70 is configured to increase the efficiency of the valve leaflet augmenter in preventing regurgitation through the valve.

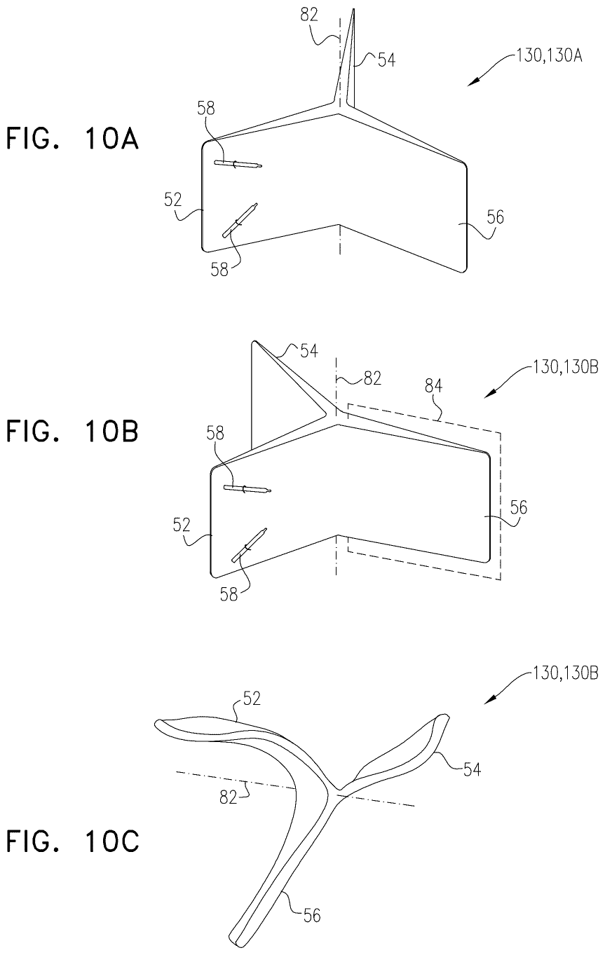

Reference is now made to FIGS. 10A-C and FIG. 11, which are schematic illustrations of a leaflet augmenter or leaflet augmentor 130 in accordance with some applications. FIG. 10A shows a variant 130A of augmenter/augmentor 130, and FIGS. 10B-C show a variant 130B.

Augmenter 130 comprises three panels, 52, 54, and 56, arranged around an axis 82 (e.g., a central axis) of the augmenter. In some applications (e.g., as shown in FIG. 10B), each panel substantially defines a respective plane 84 that is parallel to axis 82. In some applications, each panel can be somewhat curved (e.g., as shown in FIG. 10C). Each panel can be substantially circular or elliptical in shape. In some applications, each panel can be substantially rectangular in shape. The panels can include a cell growth-inducing substance. The panels can include a cell growth-inhibiting substance. Anchor(s) 58 are used to anchor panel 52 to a valve leaflet. The anchors 58 can be coupled to panel 52 closer to the edge of the panel than to axis 82 of the augmenter.

Variant 130A illustrates that, in some applications, panels 52, 54, and 56 can be distributed at 120-degree angles around axis 82, and/or can diverge at 120 degree-angles from each other. Variant 130B illustrates that, in some applications, two of the panels are oriented closer to each other, e.g., such that the angle between panels 54 and 56 is greater than 120 degrees. In FIG. 10C, augmenter 130B is shown substantially enface, such that axis 82 extends through the two-dimensional surface of the image, i.e., into and out of the plane of the page or surface on which the image is drawn.

In some applications, aspects of one or more of the variants of augmenter 30 (e.g., panel composition, panel size, panel shape, panel reinforcement, and panel anchoring) can be applied to augmenter 130. For example, the composition, support, and stiffness of panels 52, 54, and 56 can be similar to that of panels 62/32, and 64/34, mutatis mutandis. In some applications, augmenter 130 can comprise a plastic or flexible substance, e.g., a silicone or a polymer, that is shaped to define the first panel, the second panel, and the third panel. The material properties of the substance can be such that the panels can be folded or pushed together for delivery.

In some applications, augmenter 130 lacks a hinge mechanism, such that the augmenter essentially retains its three-dimensional shape after implantation during both diastole and systole. Nonetheless, augmenter 130 can be compressed for transluminal delivery—e.g., with two or more of its panels closer to each other (e.g., in contact with each other), and configured to automatically assume the configuration shown upon deployment in the heart.

In FIG. 11 is shown schematic illustrations of a system 120, in accordance with some applications, showing augmenter 130 implanted in the heart. First panel 52 is anchored to first leaflet 12 by at least one anchor 58. Anchor 58 can be as described for anchor 38, mutatis mutandis. Anchor 58 can be coupled to panel 52 closer to the edge of the panel than to axis 82 of the augmenter. When first panel 52 is anchored to first leaflet 12, it may or may not conform to the shape of the leaflet. In some applications, panel 52 can be anchored closer to root 13 of leaflet 12. When panel 52 is anchored to leaflet 12, panels 54 and 56 retain their substantially parallel relationship to axis 82.

Panel 52 can be anchored to leaflet 12 such that panel 56 extends toward ventricle 8—e.g., overhanging leaflet 12. In some cases, part of panel 56 is in contact with leaflet 12.

The upper image of FIG. 11 represents valve 10 of heart 4 during systole, and the lower image represents the valve during diastole. Features described in FIGS. 7, 8, and 9A-B can be incorporated to applications shown in FIG. 11. Augmenter 130 can be attached to leaflet 12, or in some applications, to leaflet 14. Either leaflet 12 or leaflet 14 could be a healthy leaflet or a dysfunctional leaflet. During systole, augmenter 130 bridges a potential gap between leaflets 12 and 14, such that panel 54 and/or panel 56 coapts with leaflet 14.

In some applications, panel 54 and panel 56 collectively define a face, such that after anchoring of panel 52 to leaflet 12, the face is presented as a coaptation surface to leaflet 14 during ventricular systole.

Anchor 58 can be configured to anchor panel 52 to leaflet 12 such that, during ventricular systole, panel 56 is disposed in ventricle 8 and panel 54 is disposed in atrium 6—e.g., panel 54 extends, within atrium 6, away from leaflet 12 and across an upstream surface of leaflet 14. Thus, during ventricular systole, panel 54 can be disposed against an upstream surface of leaflet 14. In some cases, a lip 17 of leaflet 14 contacts panel 56 during ventricular systole. During ventricular diastole, both panel 56 and panel 54 are typically disposed in ventricle 8.

System 120 can further comprise a tether (not shown; as in FIGS. 9A-B) coupled to and extending from, e.g., panel 52 or panel 56, and configured to tether the panel to ventricular tissue of the heart. The system/apparatus can further comprise a ventricular anchor, coupled to the tether, and configured to anchor the tether to the ventricular tissue. The ventricular tissue can be, e.g., tissue of a wall of the ventricle, or tissue of a papillary muscle. The ventricular anchor can be coupled to the tether in a manner that facilitates adjustment of tension on the tether. A winch can be used to couple the ventricular anchor to the tether, such that the tension on the tether is adjustable by actuation of the winch. The tether is configured to increase the efficiency of the valve leaflet augmenter in preventing regurgitation through the valve.

System 120 can further comprise a delivery tool for delivering the augmenter 130 to the heart 4. The delivery tool can anchor the first panel to the first leaflet while the second panel and the third panel are disposed within the delivery tool. Subsequently, the second panel and the third panel can be deployed from the delivery tool.

Reference is again made to FIGS. 1A-11. Several possible advantages of the designs shown in the present application may be identified. The augmenter or augmentor 30, 130 requires attachment to only a single valve leaflet. The design lends itself to the possibility of adapting or scaling the size of the augmenter/augmentor to an optimal size and shape for the valve being treated. In addition to being suitable for valves having incomplete coaptation, applications of the present system may be applicable to treatment of mitral annular calcification treatment, as no annular contraction is required. Some applications of augmenter/augmentor 30, 130 may have advantages in transluminal insertion as the design allows the panels to be shorter or smaller than a comparable single-panel patch. Although an optional tether is described hereinabove, it is hypothesized that, in some applications, the augmenters/augmentors described herein advantageously do not require a ventricular tether.

The systems, apparatuses, and techniques described above can be used in combination with those described in one or more of the following references, each of which is incorporated herein by reference in its entirety. For example, techniques described in one or more of the following references, each of which is incorporated by reference herein for all purposes, for attachment to ventricular tissue and/or valve leaflets, and/or for adjusting tension of a tether (e.g., a prosthetic chorda tendineae), can be used in combination with one or more of the apparatus and techniques described herein. Similarly, any of the augmenters/augmentors described herein may be used as a component of an implant (e.g., as a “patch” or “upstream assembly” of the implant) described in any of the following references: PCT Publication WO 2022/101817 to Tennenbaum et al.; U.S. Provisional Patent Application 63/113,430 to Tennenbaum et al., filed Nov. 13, 2020; U.S. Pat. No. 9,277,994 to Miller et al.; U.S. Pat. No. 9,180,007 to Reich et al.

The various systems, devices, apparatuses, etc. in this disclosure can be sterilized (e.g., with heat, radiation, ethylene oxide, hydrogen peroxide, etc.) to ensure they are safe for use with patients, and the methods herein can comprise such sterilization of the associated system, device, apparatus, etc. Furthermore, the scope of the present disclosure includes, for some applications, sterilizing one or more of any of the various systems, devices, apparatuses, etc. in this disclosure.

Any of the techniques, methods, operations, steps, etc. described or suggested herein can be performed on a living animal (e.g., human, other mammal, etc.) or on a non-living simulation, such as a cadaver, a cadaver heart, an anthropomorphic ghost, and/or a simulator device (which may include computerized and/or physical representations of body parts, tissue, etc.).

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

### Example Applications (Some Non-Limiting Examples of the Concepts Herein are Recited Below)

Example 1. A system or an apparatus, comprising an augmenter for a first leaflet of a valve of a heart, the first leaflet having a lip, the valve having a second leaflet and an annulus, the valve being disposed between an atrium and a ventricle of the heart, a root of the first leaflet being attached to the annulus, the augmenter comprising: (A) a first panel; and (B) a second panel, articulatably coupled to the first panel by an articulatable coupling, such that the augmenter is: (i) expandable by increasing an angular disposition between the first panel and the second panel, thereby increasing a dimension of an opening defined between the first panel and the second panel opposite the articulatable coupling, and (ii) collapsible by reducing the angular disposition.

Example 2. The system/apparatus according to example 1, wherein the augmenter further comprises an anchor, coupled to the first panel, and configured to anchor the first panel to the first leaflet such that: (i) the first panel is disposed against the first leaflet, (ii) the articulatable coupling is disposed closer, than the lip and the opening, to the root.

Example 3. The system/apparatus according to any one of examples 1-2, wherein the anchor is configured to anchor the first panel to the first leaflet such that the augmenter expands during ventricular systole and collapses during ventricular diastole.

Example 4. The system/apparatus according to any one of examples 1-3, wherein the anchor is configured to anchor the first panel to the first leaflet such that the opening faces the ventricle.

Example 5. The system/apparatus according to any one of examples 1-4, further comprising a flexible connector connected to the first panel and to the second panel in a manner that limits expansion of the augmenter by limiting the angular disposition between the first panel and the second panel.

Example 6. The system/apparatus according to any one of examples 1-5, wherein the anchor is coupled to the first panel closer to the articulatable coupling than to a lip of the first panel.

Example 7. The system/apparatus according to any one of examples 1-6, wherein each of the first panel and the second panel is substantially circular or elliptical in shape.

Example 8. The system/apparatus according to any one of examples 1-7, wherein each of the first panel and the second panel is substantially rectangular in shape.

Example 9. The system/apparatus according to any one of examples 1-8, wherein the first panel is sufficiently flexible to conform to a shape of the first leaflet.

Example 10. The system/apparatus according to any one of examples 1-9, wherein the first panel is dimensioned such that the anchoring, by the anchor, of the first panel to the first leaflet is such that the first leaflet overhangs beyond the lip of the first leaflet.

Example 11. The system/apparatus according to any one of examples 1-10, wherein the second panel is articulatably coupled to the first panel along an entire length of an edge of each panel.

Example 12. The system/apparatus according to any one of examples 1-11, wherein the second panel is articulatably coupled to the first panel along a partial length of an edge of each panel.

Example 13. The system/apparatus according to any one of examples 1-12, wherein the articulatable coupling comprises stitches.

Example 14. The system/apparatus according to any one of examples 1-13, wherein the articulatable coupling comprises a hinge.

Example 15. The system/apparatus according to any one of examples 1-14, wherein the articulatable coupling comprises a flexure.

Example 16. The system/apparatus according to any one of examples 1-15, wherein the anchor comprises a clip.

Example 17. The system/apparatus according to any one of examples 1-16, wherein the anchor comprises a tissue-piercing element.

Example 18. The system/apparatus according to any one of examples 1-17, wherein the augmenter is configured such that anchoring of the first panel to the first leaflet by the anchor increases an efficiency of the valve in inhibiting regurgitation of blood through the valve during ventricular systole.

Example 19. The system/apparatus according to any one of examples 1-18, wherein the first panel is dimensioned such that, upon anchoring of the first panel to the first leaflet by the anchor, the first panel overhangs beyond the lip of the first leaflet.

Example 20. The system/apparatus according to any one of examples 1-19, wherein the second panel is dimensioned to overhang beyond an edge of the first panel.

Example 21. The system/apparatus according to any one of examples 1-20, wherein the first leaflet is a flailing leaflet, and wherein the augmenter is configured to be anchored to the flailing first leaflet such that the second panel coapts with the second leaflet, thereby inhibiting flailing of the first leaflet.

Example 22. The system/apparatus according to any one of examples 1-21, wherein the second leaflet is a flailing leaflet, and wherein the augmenter is configured to be anchored to the first leaflet such that the second panel coapts with the flailing second leaflet, thereby inhibiting flailing of the second leaflet.

Example 23. The system/apparatus according to any one of examples 1-22, wherein the augmenter is configured such that, while the first panel is anchored to the first leaflet by the anchor, expansion of the augmenter reduces regurgitation of blood through the valve.

Example 24. The system/apparatus according to any one of examples 1-23, wherein the augmenter comprises a polymer that is shaped to define the first panel and the second panel.

Example 25. The system/apparatus according to any one of examples 1-24, wherein the augmenter comprises a silicone that is shaped to define the first panel and the second panel.

Example 26. The system/apparatus according to any one of examples 1-25, further comprising a tether coupled to and extending from the first panel and configured to be attached to ventricular tissue while the anchor anchors the first panel to the first leaflet.

Example 27. The system/apparatus according to example 26, wherein the anchor is a leaflet anchor, and wherein the system/apparatus further comprises a ventricular anchor, coupled to the tether, and configured to anchor the tether to the ventricular tissue while the leaflet anchor anchors the first panel to the first leaflet.

Example 28. The system/apparatus according to example 26, wherein the ventricular tissue is tissue of a wall of the ventricle.

Example 29. The system/apparatus according to example 26, wherein the ventricular tissue is tissue of a papillary muscle.

Example 30. The system/apparatus according to example 26, wherein the ventricular anchor is coupled to the tether in a manner that facilitates adjustment of tension on the tether.

Example 31. The system/apparatus according to example 30, further comprising a winch coupled to the ventricular anchor, the anchor being coupled to the tether via the winch such that the tension on the tether is adjustable by actuation of the winch.

Example 32. The system/apparatus according to any one of examples 1-31, wherein at least the first panel comprises a cell-growth-inducing material.

Example 33. The system/apparatus according to example 32, wherein the second panel comprises a cell-growth-inducing material.

Example 34. The system/apparatus according to example 32, wherein the second panel is formed from a cell growth-inhibiting material.

Example 35. The system/apparatus according to any one of examples 1-34, wherein each of the first panel and the second panel comprise a flexible fabric.

Example 36. The system/apparatus according to example 35, wherein the augmenter further comprises at least one frame.

Example 37. The system/apparatus according to example 36, wherein the at least one frame serves to reinforce the fabric.

Example 38. The system/apparatus according to example 36, wherein the fabric of each of the first panel and the second panel is disposed over the at least one frame such that the at least one frame supports the fabric.

Example 39. The system/apparatus according to example 36, wherein the at least one frame is shaped to define a hoop.

Example 40. The system/apparatus according to example 36, wherein the at least one frame has an H shape defining a crossbar, the crossbar of the H traversing a face of the panel.

Example 41. The system/apparatus according to example 36, wherein the at least one frame is positioned along at least one edge of each panel.

Example 42. The system/apparatus according to example 36, wherein the at least one frame comprises a first frame and a second frame, the first frame being articulatably coupled to the second frame.

Example 43. The system/apparatus according to example 36, wherein the at least one frame comprises a single integrated frame, having an articulation site at which the second panel is articulatably coupled to the first panel.

Example 44. The system/apparatus according to example 43, wherein the integrated frame comprises an elastic material that biases the first panel and the second panel toward assuming a predetermined angular disposition therebetween.

Example 45. The system/apparatus according to example 43, wherein the integrated frame comprises an elastic material that defines a flexure connection between the first panel and the second panel.

Example 46. The system/apparatus according to example 36, wherein the at least one frame is stitched to the fabric.

Example 47. The system/apparatus according to example 36, wherein, for each of the first panel and the second panel: (i) the flexible fabric defines a first fabric layer, disposed over a first side of the at least one frame, and (ii) the panel further comprises a second fabric layer, disposed over a second side of the at least one frame, such that the frame is sandwiched between the first fabric layer and the second fabric layer.

Example 48. The system/apparatus according to example 36, wherein the at least one frame and the fabric are dimensioned with respect to each other such that the at least one frame and the fabric are pretensioned.

Example 49. The system/apparatus according to example 48, wherein when the fabric is disposed over the at least one frame, the at least one frame provides a specific form and shape to the panel.

Example 50. The system/apparatus according to any one of examples 1-49, wherein the augmenter is biased toward the first panel and the second panel having a predetermined angular disposition therebetween.

Example 51. The system/apparatus according to example 50, wherein the augmenter is biased to collapse by reducing the angular disposition between the second panel and the first panel.

Example 52. The system/apparatus according to example 50, wherein the augmenter is biased to expand by increasing the angular disposition between the second panel and the first panel.

Example 53. The system/apparatus according to any one of examples 1-52, wherein: (A) the first panel has: (i) a lip opposite the articulatable coupling, and (ii) a lateral edge extending between the articulatable coupling and the lip of the first panel, (B) the second panel has: (i) a lip opposite the articulatable coupling, and (ii) a lateral edge extending between the articulatable coupling and the lip of the second panel, and (C) the opening is defined by the lip of the first panel and the lip of the second panel.

Example 54. The system/apparatus according to example 53, further comprising a flexible connector connected to the first panel and to the second panel in a manner that limits expansion of the augmenter by limiting the angular disposition between the first panel and the second panel.

Example 55. The system/apparatus according to example 54, wherein the flexible connector is located opposite the articulatable coupling, extending between the first panel and the second panel.

Example 56. The system/apparatus according to example 55, wherein the flexible connector connects the lip of the first panel and the lip of the second panel.

Example 57. The system/apparatus according to example 56, wherein the flexible connector connects a substantially central part of the lip of the first panel to a substantially central part of the lip of the second panel.

Example 58. The system/apparatus according to example 56, wherein: (A) the flexible connector is a first flexible connector, (B) the augmenter further comprises a second flexible connector, (C) the first flexible connector connects a first lateral part of the lip of the first panel to a first lateral part of the lip of the second panel, and (D) the second flexible connector connects: a second lateral part of the lip of the first panel, substantially opposite the first lateral part of the lip of the first panel, to a second lateral part of the lip of the second panel, substantially opposite the first lateral part of the lip of the second panel.

Example 59. The system/apparatus according to example 55, wherein the flexible connector connects (i) a site on the first panel partway between the articulatable coupling and the lip of the first panel, and (ii) a site on the second panel partway between the articulatable coupling and the lip of the second panel.

Example 60. The system/apparatus according to example 54, wherein the flexible connector connects the lateral edge of the first panel to the lateral edge of the second panel.

Example 61. The system/apparatus according to example 54, wherein the flexible connector comprises a flexible third panel extending between the first panel and the second panel.

Example 62. The system/apparatus according to example 54, wherein the flexible connector includes a cell-growth-inhibiting substance.

Example 63. The system/apparatus according to example 54, wherein the flexible connector includes a cell-growth-promoting substance.

Example 64. The system/apparatus according to example 61, wherein the flexible third panel connects the lip of the first panel to the lip of the second panel.

Example 65. The system/apparatus according to example 61, wherein the third panel comprises at least one of a fabric and a net.

Example 66. The system/apparatus according to example 61, wherein the anchor is configured to anchor the first panel to the first leaflet such that the third panel coapts with the second leaflet during systole.

Example 67. The system/apparatus according to example 61, wherein the anchor is configured to anchor the first panel to the first leaflet such that the third panel faces the ventricle during systole.

Example 68. The system/apparatus according to example 61, wherein: (i) the lateral edge of the first panel is a first lateral edge of the first panel, (ii) the lateral edge of the second panel is a first lateral edge of the second panel, substantially aligned with the first lateral edge of the first panel, (iii) the first panel has a second lateral edge, substantially opposite the first lateral edge of the first panel, and extending between the articulatable coupling and the lip of the first panel, (iv) the second panel has a second lateral edge substantially opposite the first lateral edge of the second panel, extending between the articulatable coupling and the lip of the second panel, and substantially aligned with the second lateral edge of the first panel, (v) the third panel extends between the first lateral edge of the first panel and the first lateral edge of the second panel, and (vi) the augmenter further comprises a flexible fourth panel, extending between the second lateral edge of the first panel and the second lateral edge of the second panel.

Example 69. The system/apparatus according to example 68, wherein the third panel and the fourth panel comprise folds configured to collapse inward when the opening collapses.

Example 70. The system/apparatus according to example 54, wherein the flexible connector comprises at least one connector element selected from a group consisting of: a wire, and a thread.

Example 71. The system/apparatus according to example 71, wherein the flexible connector comprises exactly one connector element selected from the group.

Example 72. The system/apparatus according to example 53, wherein the anchor is coupled to the first panel closer to the articulatable coupling than to the lip of the first panel.

Example 73. The system/apparatus according to any one of examples 1-72, wherein the anchor comprises a toggle anchor.

Example 74. The system/apparatus according to example 73, wherein the toggle anchor has a blunt end and a sharp end and is configured to be advanced through the first leaflet sharp-end-first.

Example 75. The system/apparatus according to example 73, wherein the anchor is coupled to the first panel by a flexible thread.

Example 76. The system/apparatus according to example 73, wherein the anchor is a first anchor, and wherein the augmenter further comprises a second anchor that is coupled to the first panel, and that is configured to anchor the first panel to the first leaflet.

Example 77. The system/apparatus according to example 73, wherein the first anchor comprises a first toggle anchor, and the second anchor comprises a second toggle anchor.

Example 78. The system/apparatus according to any one of examples 1-77, wherein the augmenter is configured such that, while (i) the first panel is anchored to the first leaflet, and (ii) the articulatable coupling is disposed closer to the root, than the lip and the opening, blood flow in the heart transitions the augmenter toward its expanded configuration during ventricular systole, and toward its collapsed configuration during ventricular diastole.

Example 79. The system/apparatus according to example 78, further comprising a flexible connector connected to the first panel and to the second panel in a manner that limits expansion of the augmenter during ventricular systole.

Example 80. The system/apparatus according to any one of examples 1-79, wherein the system/apparatus is sterilized.

Example 81. A system or an apparatus, comprising an augmenter for a first leaflet of a valve of a heart, the first leaflet having a lip, the valve being disposed between an atrium and a ventricle of the heart and having a second leaflet, the system/apparatus comprising: (A) a first panel; (B) a second panel; (C) a third panel; and (D) an anchor, wherein each of the panels: (i) has a respective outer edge, (ii) extends inwardly from the outer edge, such that the first, second, and third panels converge at an axis of the augmenter, and (iii) defines a panel plane that is substantially parallel with the axis; and wherein the anchor is configured to anchor the first panel to the first leaflet.

Example 82. The system/apparatus according to example 81, wherein the system/apparatus is sterilized.

Example 83. The system/apparatus according to any one of examples 81-82, wherein the augmenter comprises a polymer that is shaped to define the first panel, the second panel, and the third panel.

Example 84. The system/apparatus according to any one of examples 81-83, wherein the augmenter comprises a silicone that is shaped to define the first panel, the second panel, and the third panel.

Example 85. The system/apparatus according to any one of examples 81-84, wherein the anchor is configured to attach the first panel to the first leaflet such that the second panel extends away from the first panel toward the second leaflet.

Example 86. The system/apparatus according to any one of examples 81-85, wherein the anchor is configured to anchor the first panel to the first leaflet such that the third panel extends away from the first panel toward the ventricle.

Example 87. The system/apparatus according to any one of examples 81-86, wherein the anchor is coupled to the first panel closer to the edge of the first panel than to the axis of the augmenter.

Example 88. The system/apparatus according to any one of examples 81-87, wherein each of the first panel, the second panel, and the third panel is substantially circular or elliptical in shape.

Example 89. The system/apparatus according to any one of examples 81-88, wherein each of the first panel, the second panel, and the third panel is substantially rectangular in shape.

Example 90. The system/apparatus according to any one of examples 81-89, wherein the first panel is sufficiently flexible to conform to a shape of the first leaflet.

Example 91. The system/apparatus according to any one of examples 81-90, wherein the first panel is dimensioned such that the anchoring, by the anchor, of the first panel to the first leaflet is such that the third panel overhangs beyond the lip of the first leaflet.

Example 92. The system/apparatus according to any one of examples 81-91, wherein: (A) the second panel and the third panel collectively define a face, and (B) the anchor is configured to anchor the first panel to the first leaflet such that: (i) the first panel is disposed against the first leaflet, and (ii) the face is presented as a coaptation surface to the second leaflet.

Example 93. The system/apparatus according to example 92, wherein the anchor is configured to anchor the first panel to the first leaflet such that the third panel is disposed in the ventricle.

Example 94. The system/apparatus according to example 92, wherein the anchor is configured to anchor the first panel to the first leaflet such that, during ventricular systole, the second panel extends, within the atrium, away from the first panel and across an upstream surface of the second leaflet.

Example 95. The system/apparatus according to example 92, wherein the anchor is configured to anchor the first panel to the first leaflet such that, during ventricular systole, the second panel is disposed against an upstream surface of the second leaflet.

Example 96. The system/apparatus according to example 92, wherein the anchor is configured to anchor the first panel to the first leaflet such that, during ventricular systole, a lip of the second leaflet contacts the third panel.

Example 97. The system/apparatus according to any one of examples 81-96, further comprising a tether coupled to and extending from the first panel and configured to tether the first panel to ventricular tissue of the heart.

Example 98. The system/apparatus according to example 97, wherein the anchor is a leaflet anchor, and wherein the system/apparatus further comprises a ventricular anchor, coupled to the tether, and configured to anchor the tether to the ventricular tissue while the leaflet anchor anchors the first panel to the first leaflet.

Example 99. The system/apparatus according to example 97, wherein the ventricular tissue is tissue of a wall of the ventricle.

Example 100. The system/apparatus according to example 97, wherein the ventricular tissue is tissue of a papillary muscle.

Example 101. The system/apparatus according to example 97, wherein the ventricular anchor is coupled to the tether in a manner that facilitates adjustment of tension on the tether.

Example 102. The system/apparatus according to example 97, further comprising a winch coupled to the ventricular anchor, the anchor being coupled to the tether via the winch such that the tension on the tether is adjustable by actuation of the winch.

Example 103. The system/apparatus according to any one of examples 81-102, wherein at least the first panel comprises a cell-growth-inducing material.

Example 104. The system/apparatus according to example 103, wherein the second panel includes a cell-growth-inducing material.

Example 105. The system/apparatus according to example 103, wherein the third panel includes a cell growth-inducing material.

Example 106. The system/apparatus according to example 103, wherein the second panel includes a cell-growth-inhibiting material.

Example 107. The system/apparatus according to example 103, wherein the third panel includes a cell growth-inhibiting material.

Example 108. The system/apparatus according to any one of examples 81-107, wherein each of the first panel, the second panel, and the third panel comprises a flexible fabric.

Example 109. The system/apparatus according to example 108, wherein the augmenter further comprises a frame.

Example 110. The system/apparatus according to example 109, wherein the frame serves to reinforce the fabric.

Example 111. The system/apparatus according to example 109, wherein the fabric of each of the first panel, the second panel, and the third panel is disposed over the frame such that the frame supports the fabric.

Example 112. The system/apparatus according to example 109, wherein, for each of the first, second, and third panels, the frame extends along the outer edge the panel, substantially parallel with the axis.

Example 113. The system/apparatus according to example 109, wherein the frame comprises a single integrated frame defining an outline of the first panel, the second panel, and the third panel.

Example 114. The system/apparatus according to example 109, wherein the integrated frame comprises an elastic material that biases the first panel, the second panel, and the third panel toward assuming a predetermined angular disposition with respect to each other.

Example 115. The system/apparatus according to example 109, wherein the integrated frame comprises an elastic material.

Example 116. The system/apparatus according to example 109, wherein the frame is stitched to the fabric.

Example 117. The system/apparatus according to example 109, wherein, for each of the first panel, the second panel, and the third panel: (i) the flexible fabric defines a first fabric layer, disposed over a first side of the frame, and (ii) the panel further comprises a second fabric layer, disposed over a second side of the frame, such that the frame is sandwiched between the first fabric layer and the second fabric layer.

Example 118. The system/apparatus according to example 109, wherein the frame and the fabric are sized with respect to each other such that the frame and the fabric are pretensioned.

Example 119. The system/apparatus according to example 109, wherein when the fabric is disposed over the frame, the frame provides a specific form and shape to the first panel, the second panel, and the third panel.

Example 120. A method for use with a simulated valve of a simulated heart, the simulated valve having a first leaflet and a second leaflet, and being disposed between an atrium and a ventricle of the heart, the method comprising: (A) transluminally advancing an augmenter to the simulated heart, the augmenter including: (i) a first panel, (ii) a second panel articulatably coupled to the first panel, and (iii) an anchor; and (B) anchoring the first panel to the first leaflet using the anchor, such that: (i) the first panel of the augmenter is disposed against the first leaflet, (ii) the second panel, in response to blood flow in the simulated heart: (a) deflects away from the first panel and toward the second leaflet during ventricular systole; and (b) deflects away from the second leaflet and toward the first panel during ventricular diastole.

Example 121. The method according to example 120, wherein anchoring the first panel to the first leaflet comprises anchoring the first panel to the first leaflet such that the second panel, in response to blood flow in the heart, coapts with the second leaflet during ventricular systole, thereby reducing regurgitation through the simulated valve.

Example 122. The method according to any one of examples 120-121, wherein deflection of the second panel during ventricular systole leads to the second panel coapting with the second leaflet.

Example 123. The method according to any one of examples 120-122, further comprising tethering the first panel to ventricular tissue using a tether connected to the first panel.

Example 124. The method according to any one of examples 120-123, further comprising adjusting tension on the tether.

Example 125. The method according to any one of examples 120-124, wherein tethering the first panel to the ventricular tissue comprises anchoring the tether to the ventricular tissue using a tissue anchor attached to the tether.

Example 126. The method according to any one of examples 120-125, further comprising coupling the tissue anchor to the tether via a winch.

Example 127. The method according to any one of examples 120-126, further comprising adjusting tension on the tether by actuating the winch.

Example 128. The method according to any one of examples 120-127, further comprising attaching a flexible third panel between the first panel and the second panel.

Example 129. The method according to any one of examples 120-128, wherein deflection of the second panel during ventricular systole is accompanied by the flexible third panel coapting with the second leaflet.

Example 130. A method for use with a simulated valve of a simulated heart, the simulated valve having a first leaflet and a second leaflet, and being disposed between an atrium and a ventricle of the heart, the method comprising: (A) transluminally advancing an augmenter to the simulated heart, the augmenter including: (i) a first panel, (ii) a second panel, (iii) a third panel, and (iv) an anchor; and (B) anchoring the first panel to the first leaflet, wherein each of the panels: (i) has a respective outer edge, (ii) extends inwardly from the outer edge, such that the first, second, and third panels converge at an axis of the augmenter, and (iii) defines a respective panel plane that is substantially parallel with the axis.

Example 131. The method according to example 130, wherein: (i) anchoring the first panel to the first leaflet comprises anchoring the first panel to the first leaflet while the second panel and the third panel are disposed within a delivery tool, and (ii) the method further comprises subsequently deploying the second panel and the third panel from the delivery tool.

Example 132. The method according to any one of examples 130-131, wherein deploying the second panel and the third panel from the delivery tool comprises deploying the second panel and the third panel from the delivery tool such that the second panel and the third panel deflect away from each other.

Example 133. A system or an apparatus, comprising a leaflet augmenter for use at a valve of a heart, the valve having at least a first leaflet and a second leaflet, the system/apparatus comprising: (i) a first panel, and a second panel articulatably connected to the first panel; and (ii) a tissue-piercing element coupled to the first panel and configured to anchor the first panel to the first leaflet such that, during ventricular systole, closure of the valve is accompanied by articulation of the second panel away from the first panel and toward the second leaflet.

1-20. (canceled)

21. An apparatus, for use with a valve of a heart of a subject, the apparatus comprising:
an augmenter, having an upstream-facing aspect that is shaped as an arc, the augmenter comprising:
an anchor configured to anchor the augmenter to the valve;
a first panel;
a second panel, attached to the first panel along the arc, the augmenter defining a downstream-facing opening between the first panel and the second panel; and
a delivery tool configured to anchor the upstream-facing aspect of the augmenter to the valve such that:
the first panel coapts with a first leaflet of the valve,
the second panel coapts with a second leaflet of the valve, and
the downstream-facing opening expands and contracts responsively to a cardiac cycle of the heart, facilitated by movement of the second panel with respect to the first panel.

22. The apparatus according to claim 21, wherein the anchor is configured to anchor the augmenter to the valve such that the augmenter expands during ventricular systole and collapses during ventricular diastole.

23. The apparatus according to claim 21, wherein the anchor is configured to anchor the augmenter to the valve such that the opening faces a ventricle downstream of the valve.

24. The apparatus according to claim 21, wherein the first panel is sufficiently flexible to conform to a shape of the first leaflet.

25. The apparatus according to claim 21, wherein the anchor comprises a tissue-piercing element.

26. The apparatus according to claim 21, wherein the augmenter comprises a polymer that is shaped to define the first panel and the second panel.

27. The apparatus according to claim 21, wherein the augmenter comprises a silicone that is shaped to define the first panel and the second panel.

28. The apparatus according to claim 21, wherein:
the anchor is a valve anchor, and
the apparatus further comprises:
a tether, coupled to and extending from the first panel; and
a ventricular anchor, coupled to the tether,
the delivery tool being configured to tether the first panel in a ventricle downstream of the valve by anchoring the ventricular anchor to ventricular tissue.

29. The apparatus according to claim 28, wherein the ventricular anchor is coupled to the tether in a manner that facilitates adjustment of tension on the tether.

30. The apparatus according to claim 29, further comprising a winch coupled to the ventricular anchor, the anchor being coupled to the tether via the winch such that the tension on the tether is adjustable by actuation of the winch.

31. The apparatus according to claim 28, wherein at least the first panel comprises a cell-growth-inducing material.

32. The apparatus according to claim 28, wherein the second panel comprises a cell-growth-inducing material.

33. The apparatus according to claim 28, wherein the second panel is formed from a cell growth-inhibiting material.

34. The apparatus according to claim 28, wherein each of the first panel and the second panel comprise a flexible fabric.

35. The apparatus according to claim 34, wherein the augmenter further comprises a frame that extends along the arc, the fabric being disposed over the frame such that the frame supports the fabric.

36. The apparatus according to claim 35, wherein the frame is a first frame that supports the fabric of the first panel being disposed over the first frame, and wherein the augmenter further comprises a second frame, coupled to the first frame along the arc, the fabric of the second panel being disposed over the second frame.

37. The apparatus according to claim 35, wherein the frame is a single integrated frame over which the fabric of the first panel and the fabric of the second panel are disposed, and having an articulation site at which the second panel is articulatably coupled to the first panel.

38. The apparatus according to claim 37, wherein the single integrated frame comprises an elastic material that biases the first panel and the second panel toward assuming a predetermined angular disposition therebetween.

39. The apparatus according to claim 35, wherein the fabric is arranged as:
a first fabric layer, disposed over a first side of the frame, and
a second fabric layer, disposed over a second side of the frame, such that the frame is sandwiched between the first fabric layer and the second fabric layer.
